(19) United States
(12) Patent Application Publication
(10) Pub. No.: US 2024/0140971 A1
(43) Pub. Date: May 2, 2024

(54) PSMA TARGETED RADIOHALOGENATED UREAS FOR CANCER RADIOTHERAPY

(71) Applicants: The Johns Hopkins University, Baltimore, MD (US); Duke University, Durham, NC (US)

(72) Inventors: Martin G. Pomper, Baltimore, MD (US); Ronnie C. Mease, Fairfax, VA (US); Ying Chen, Lutherville-Timonium, MD (US); Sangeeta Ray, Ellicott City, MD (US); Michael Zalutsky, Chapel Hill, NC (US); Ganesan Vaidyanathan, Chapel Hill, NC (US)

(21) Appl. No.: 18/209,798

(22) Filed: Jun. 14, 2023

Related U.S. Application Data
(63) Continuation of application No. 15/769,865, filed on Apr. 20, 2018, now abandoned.
(60) Provisional application No. 62/402,284, filed on Sep. 30, 2016.
(60) Provisional application No. 62/245,022, filed on Oct. 22, 2015.

(51) Int. Cl. C07F 13/00; A61P 35/00

(52) U.S. Cl. CPC C07F 13/00; A61P 35/00; C07B 2200/05

(57) ABSTRACT

PSMA binding scaffolds with radioiodinated, radiobrominated and radioastatinated labeled prosthetic groups are disclosed. Pharmaceutical compositions and methods of treating PSMA expressing cells or tumors also are disclosed.

### BACKGROUND

The prostate-specific membrane antigen (PSMA) is a type II integral membrane protein expressed on the surface of prostate tumors, particularly in castrate-resistant, advanced and metastatic disease (Huang, 2004; Schuelke, 2003). PSMA also is expressed in neovascular endothelium of most solid tumors, such as lung, colon, pancreatic, renal carcinoma and skin melanoma, but not in normal vasculature (Liu, 1997; Chang, 1999), which makes it an excellent target for imaging and targeted therapy of these cancers. Prostate cancer is the leading cancer in the U.S. population and the second leading cause of cancer death in men. Therapy for locally advanced disease remains contentious and an increasing number of disparate options are available. Over the past years a variety of high affinity, radiohalogenated urea-based PSMA inhibitors that selectively image prostate tumors in experimental models have been synthesized. Because of the favorable pharmacokinetic profile of this class of compounds, i.e., low nonspecific binding, lack of metabolism in vivo and reasonable tumor residence times, the imaging studies have been extended to molecular radiotherapy. This will be in analogy with radioimmunotherapy (RIT), which has proved remarkably successful in the treatment of lymphoma with two commercial products routinely integrated into clinical practice. However, RIT is fraught with similar difficulties to the use of radiolabeled antibodies for imaging, including prolonged circulation times, unpredictable biological effects and the occasional need for pre-targeting strategies. Furthermore, antibodies may have less access to tumors than low molecular weight agents, which can be manipulated pharmacologically. Therefore, a need remains for low molecular weight compounds with high binding affinity to PSMA for cancer radiotherapy.

### SUMMARY

In some aspects, the presently disclosed subject matter provides compounds of formula (I):

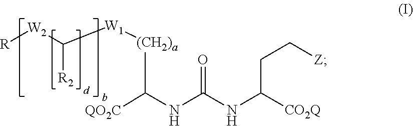

wherein: Z is tetrazole or CO₂Q; Q is H or a protecting group; a is an integer selected from the group consisting of 0, 1, 2, 3, 4, 5, 6, 7, and 8; W₁ is selected from the group consisting of —C(═O)—NR₁—, —NR₁—C(═O)—, and —S—; each R₁ is independently H or a C₁-C₆ alkyl; each R₂ is independently H or —COOR₃; each R₃ is independently H, C₁-C₆ alkyl, C₆-C₁₂ aryl or C₄-C₁₆ alkylaryl; b is an integer selected from the group consisting of 0, 1, 2, and 3; d is an integer selected from the group consisting of 1, 2, 3, 4, 5, 6, 7, and 8; each W₂ is independently selected from the group consisting of —C(═O)—NR₁— and —NR₁—C(═O)—; R is selected from the group consisting of:

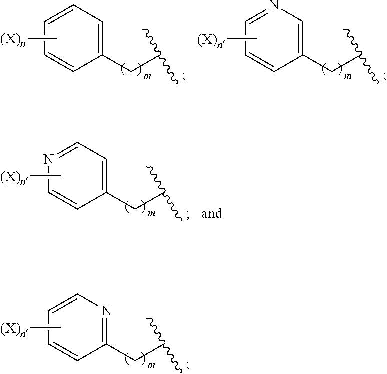

wherein X is selected from the group consisting of iodine, astatine, bromine, a radioisotope of iodine, a radioisotope of astatine, a radioisotope of bromine, Sn(R₄)₃, Si(R₄)₃, Hg(R₄), B(OH)₂, —NHNH₂, —CH₂—NH—C(═NH)—NH₂; R₄ is C₁-C₆ alkyl; m is an integer selected from the group consisting of 0, 1, 2, 3, 4, 5, 6, 7, and 8; n is an integer selected from the group consisting of 1, 2, 3, 4, and 5; n′ is an integer selected from the group consisting of 1, 2, 3, and 4; and stereoisomers and pharmaceutically acceptable salts thereof.

In certain aspects, the presently disclosed subject matter provides a method for treating one or more PSMA expressing tumors or cells, the method comprising contacting the one or more PSMA expressing tumors or cells with an effective amount of a compound of formula (I).

In other aspects, the presently disclosed subject matter provides a pharmaceutical composition comprising a pharmaceutically acceptable carrier and a compound of formula (I).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows three PSMA binding scaffolds: the lysine-glutamate urea 1, cysteine-glutamate urea 2, and glutamate-glutamate urea 3;

FIG. 2 shows examples of lysine glutamate urea and glutamate glutamate urea compounds for radiotherapy;

FIG. 3 shows the cysteine-glutamate urea scaffold used for PSMA binding and imaging for over 10 years: C-11 labeled DCMC (Pomper et al., 2002; Foss et al., 2005), F-18 labeled DCFBC (Mease et al., 2008; Cho et al., 2012) both for PET imaging with the latter currently in use in patients, and 1-125 labeled DCIBC (Dusich 2008) for SPECT imaging and or radiotherapy;

FIG. 4A and FIG. 4B show preparative HPLC chromatograms for purified [²¹¹At] YC-I-27 after standing one hour in ethanol; (FIG. 4A) radio-HPLC peak; and (FIG. 4B) UV trace at λ=254 nm; no UV peak was observed due to the high specific activity of ²¹¹At;

FIG. 5A and FIG. 5B show (FIG. 5A) the binding specificity of [²¹¹At]YC-I-27 in PSMA positive cells and (FIG. 5B) the cell kill due to [²¹¹At]YC-I-27 vs. free [²¹¹At]astatide; and

FIG. 6A, FIG. 6B, FIG. 6C, and FIG. 6D show comparative examples of PSMA specific tumor growth inhibition from 3 to 19 days post-injection in (FIG. 6A) PSMA+(PIP) tumors untreated; (FIG. 6B) PSMA+(PIP) tumors treated with [²¹¹At]YC-I-27 (20 μCO; (FIG. 6C) PSMA−(Flu) tumors untreated; (FIG. 6D) PSMA−(Flu) tumors treated with [²¹¹At]YC-1-27(20 μCi).

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures.

Therefore, it is to be understood that the presently disclosed subject matter is not to be limited to the specific embodiments disclosed and that modifications and other embodiments are intended to be included within the scope of the appended claims.

### I. Psma Targeted Radiohalogenated Ureas for Cancer Radiotherapy

The presently disclosed subject matter provides PSMA binding ureas with radioiodinated, radiobrominated and At-211 labeled prosthetic groups. Also disclosed is the first example of an At-211 labeled PSMA inhibitor, which exhibits PSMA specific tumor growth inhibition. In some embodiments, the presently disclosed radiohalogenated ureas bind to PSMA with extremely high affinity, which results from the agent completely occupying the binding cavity.

### A. Compounds of Formula (I)

Accordingly, in some embodiments, the presently disclosed subject matter provides a compound of formula (I):

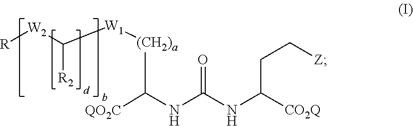

wherein: Z is tetrazole or CO₂Q; Q is H or a protecting group; a is an integer selected from the group consisting of 0, 1, 2, 3, 4, 5, 6, 7, and 8; W₁ is selected from the group consisting of —C(═O)—NR₁—, —NR₁—C(═O)—, and —S—; each R₁ is independently H or a C₁-C₆ alkyl; each R₂ is independently H or —COOR₃; each R₃ is independently H, C₁-C₆ alkyl, C₆-C₁₂ aryl or C₄-C₁₆ alkylaryl; b is an integer selected from the group consisting of 0, 1, 2, and 3;
d is an integer selected from the group consisting of 1, 2, 3, 4, 5, 6, 7, and 8;
each W₂ is independently selected from the group consisting of —C(═O)—NR₁— and —NR₁—C(═O)—; R is selected from the group consisting of:

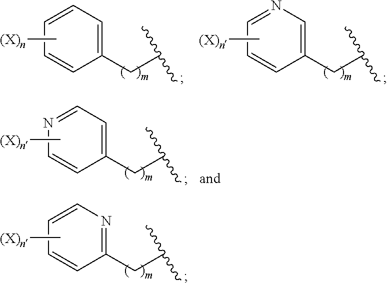

wherein X is selected from the group consisting of iodine, astatine, bromine, a radioisotope of iodine, a radioisotope of astatine, a radioisotope of bromine, Sn(R₄)₃, Si(R₄)₃, Hg(R₄), B(OH)₂, —NHNH₂, —CH₂—NH—C(═NH)—NH₂; R₄ is C₁-C₆ alkyl; m is an integer selected from the group consisting of 0, 1, 2, 3, 4, 5, 6, 7, and 8; n is an integer selected from the group consisting of 1, 2, 3, 4, and 5; n′ is an integer selected from the group consisting of 1, 2, 3, and 4; and stereoisomers and pharmaceutically acceptable salts thereof.

With regard to the composition of matter subject matter, formula (I) does not include compounds disclosed in WO 2008/058192, WO 2010/014933, and U.S. Pat. No. 7,408,079, each of which is incorporated herein by reference in their entirety. More particularly, the following compounds are expressly disclaimed from the composition of matter claims in the present application:

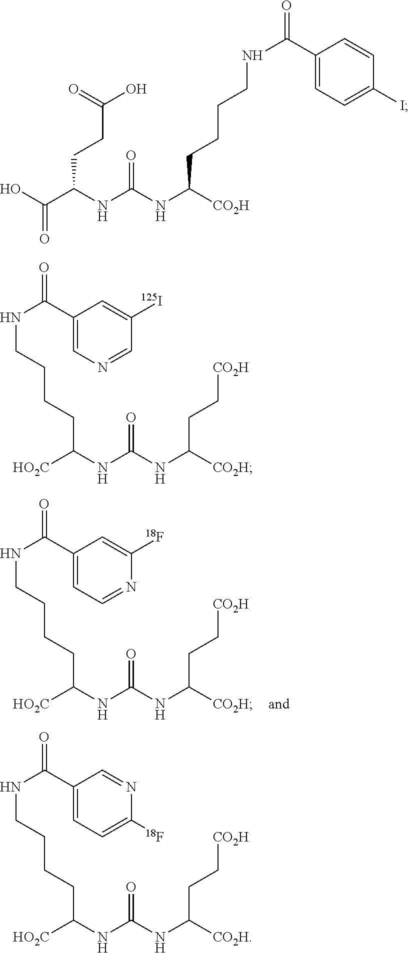

In particular embodiments, the compound of formula (I) is selected from the group consisting of:

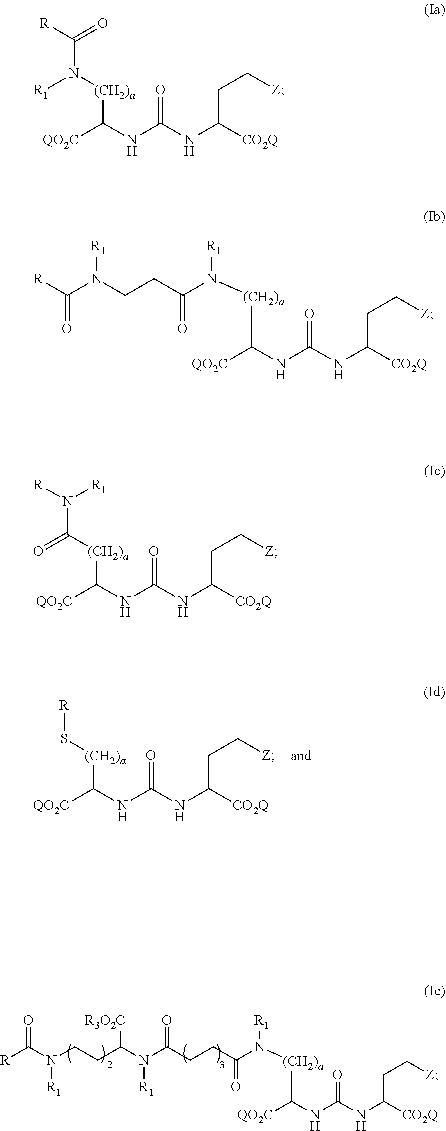

wherein Z, Q, R, R₁, R₃, a are defined as above; and stereoisomers and pharmaceutically acceptable salts thereof.

In further embodiments, the compound of formula (I) is selected from the group consisting of:

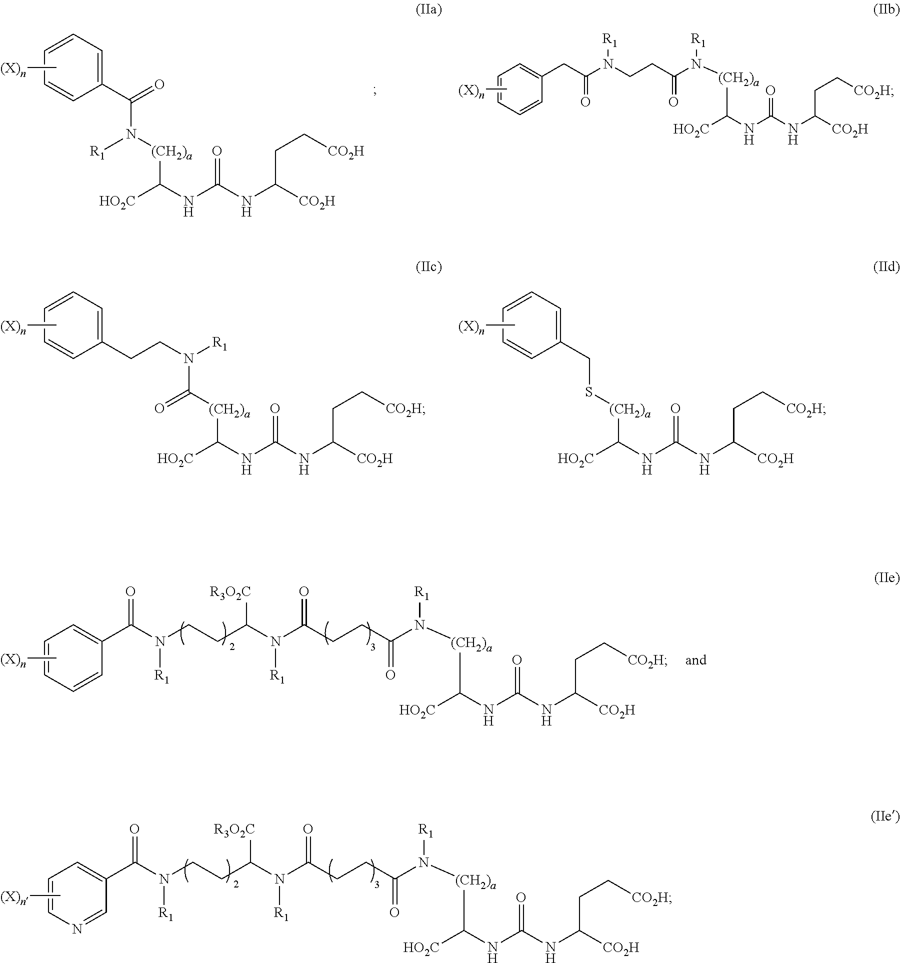

wherein Z, Q, R, R₁, R₃, X, a and n are defined hereinabove; and stereoisomers and pharmaceutically acceptable salts thereof. In some embodiments, X is selected from the group consisting of ¹²⁵I, ¹²³I, ¹³¹I, ²¹¹At, ⁷⁷Br, and ^{80m}Br.

In particular embodiments, the compound of formula (I) is selected from the group consisting of:

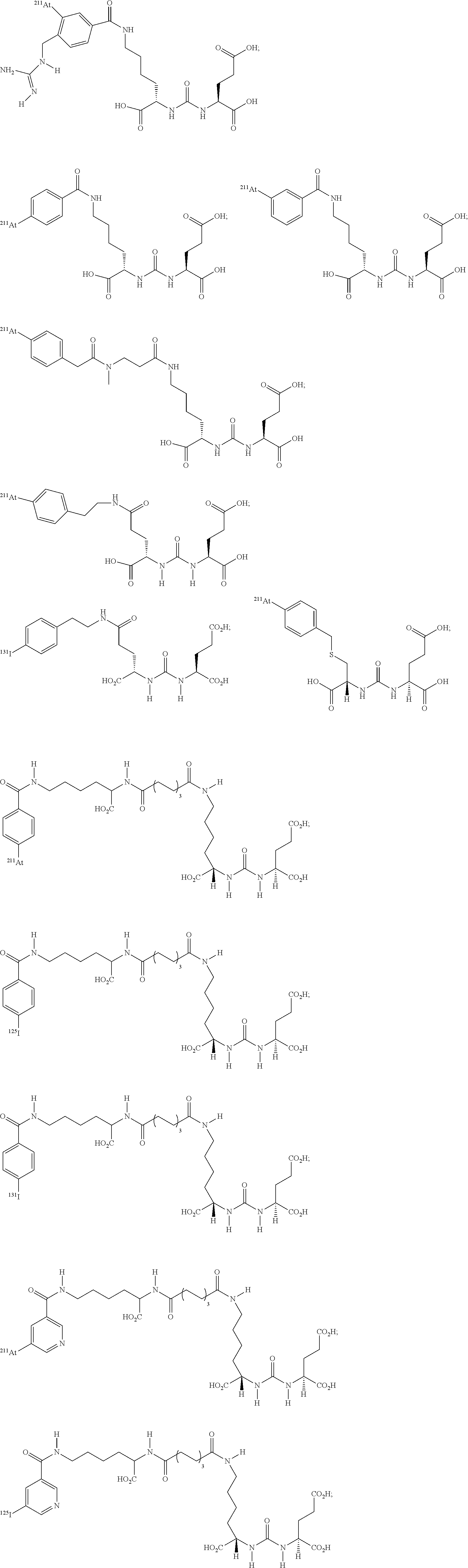

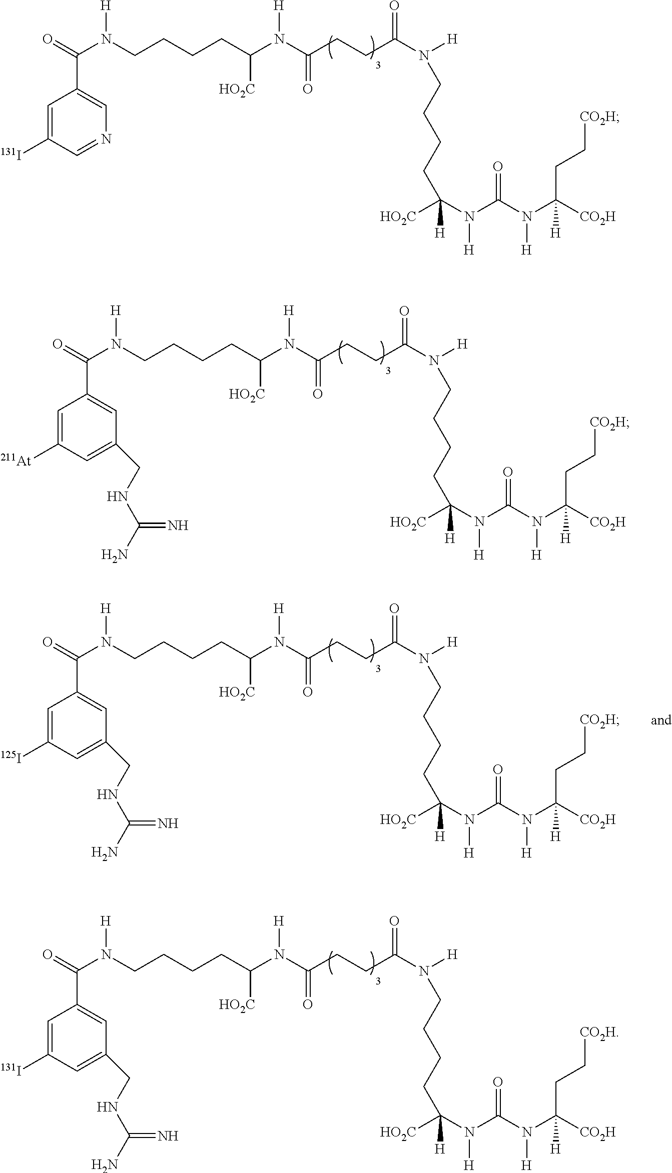

In yet more particular embodiments, the compound of formula (I) is selected from the group consisting of:

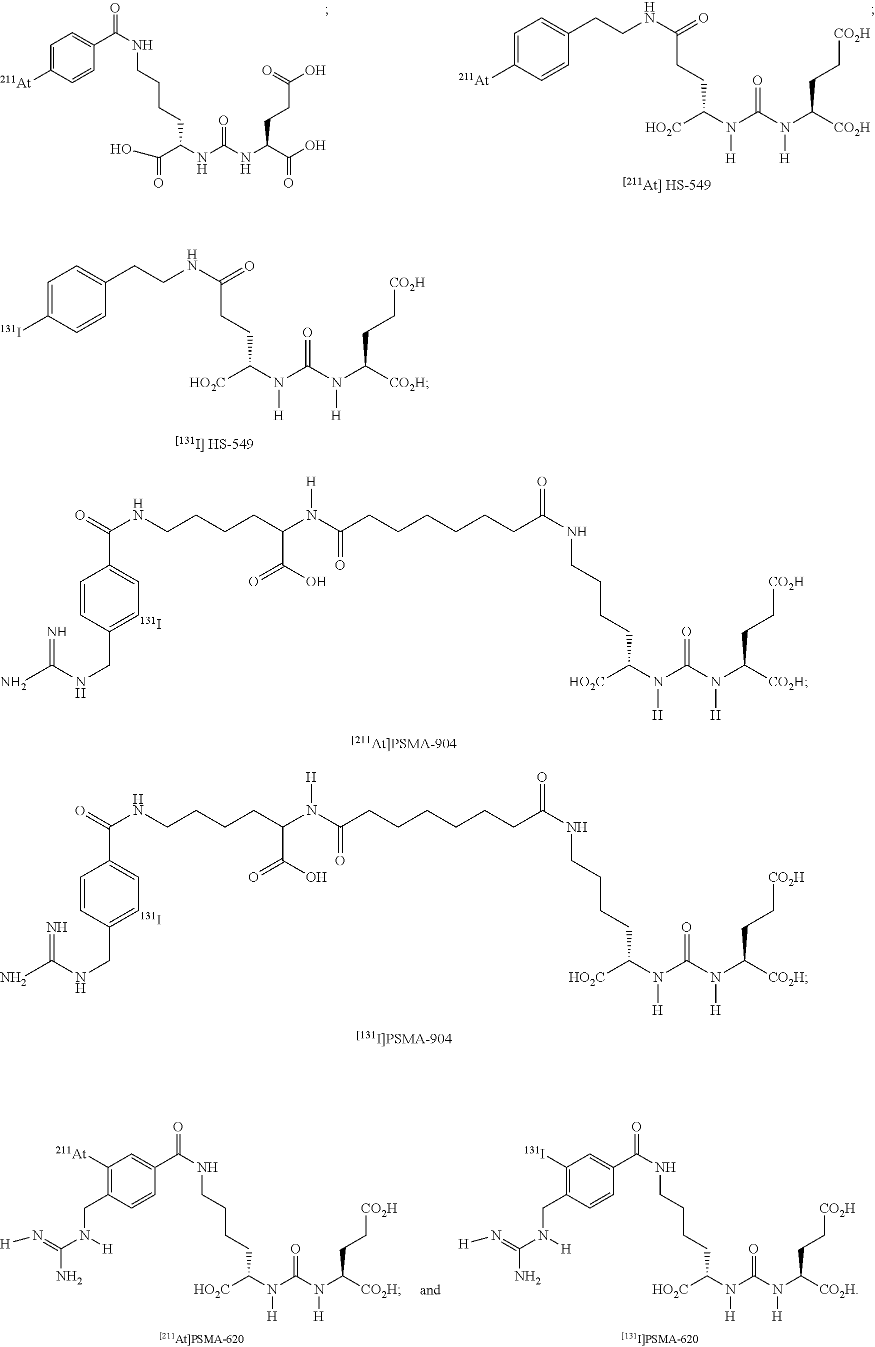

B. Methods of Using Compounds of Formula (I) for Treating a PSMA-expressing Tumor or Cell

In some embodiments, the presently disclosed subject matter provides a method for treating one or more PSMA expressing tumors or cells, the method comprising contacting the one or more PSMA expressing tumors or cells with an effective amount of a compound of formula (I), the compound of formula (I) comprising:

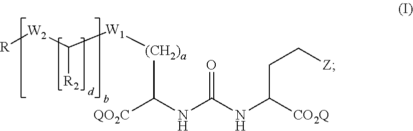

wherein: Z is tetrazole or CO₂Q; Q is H or a protecting group; a is an integer selected from the group consisting of 0, 1, 2, 3, 4, 5, 6, 7, and 8; W₁ is selected from the group consisting of —C(═O)—NR₁—, —NR₁—C(═O)—, and —S—; each R₁ is independently H or a C₁-C₆ alkyl; each R₂ is independently H or —COOR₃; each R₃ is independently H or a C₁-C₆ alkyl; b is an integer selected from the group consisting of 0, 1, 2, and 3; d is an integer selected from the group consisting of 1, 2, 3, 4, 5, 6, 7, and 8; each W₂ is independently selected from the group consisting of —C(═O)—NR₁— and —NR₁—C(═O)—; R is:

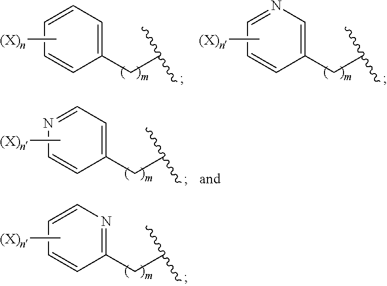

wherein X is selected from the group consisting of Sn(R₄)₃, Si(R₄)₃, Hg(R₄), B(OH)₂, —NHNH₂, —CH₂—NH—C(═NH)—NH₂, a radioisotope of iodine, a radioisotope of astatine, or a radioisotope of bromine; R₄ is C₁-C₆ alkyl; m is an integer selected from the group consisting of 0, 1, 2, 3, 4, 5, 6, 7, and 8; n is an integer selected from the group consisting of 1, 2, 3, 4, and 5; n′ is an integer selected from the group consisting of 1, 2, 3, and 4; and stereoisomers and pharmaceutically acceptable salts thereof.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. The treatment, administration, or therapy can be consecutive or intermittent. Consecutive treatment, administration, or therapy refers to treatment on at least a daily basis without interruption in treatment by one or more days. Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

“Contacting” means any action which results in at least one compound comprising the treating agent of the presently disclosed subject matter physically contacting at least one or more PSMA-expressing tumors or cells. Contacting can include exposing the PSMA-expressing tumors or cells to the compound in an amount sufficient to result in contact of at least one compound with at least one PSMA-expressing tumor or cell.

By “agent” is meant a compound of Formula (I), including compounds of formula (Ia), (Ib), (Ic), (Id), (Ie), (Ha), (IIb), (IIc), (IId), (IIe), and (IIe′) or another agent, e.g., a peptide, nucleic acid molecule, or other small molecule compound administered in combination with a compound of Formula (I).

More particularly, the term “therapeutic agent” means a substance that has the potential of affecting the function of an organism. Such an agent may be, for example, a naturally occurring, semi-synthetic, or synthetic agent. For example, the therapeutic agent may be a drug that targets a specific function of an organism. A therapeutic agent also may be an antibiotic or a nutrient. A therapeutic agent may decrease, suppress, attenuate, diminish, arrest, or stabilize the development or progression of disease, disorder, or condition in a host organism.

In general, the “effective amount” of an active agent refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

Formula (I) does not include compounds disclosed in WO 2008/058192, WO 2010/014933, and U.S. Pat. No. 7,408,079, each of which is incorporated herein by reference in their entirety. More particularly, the following compounds are expressly disclaimed from the treatment claims in the present application:

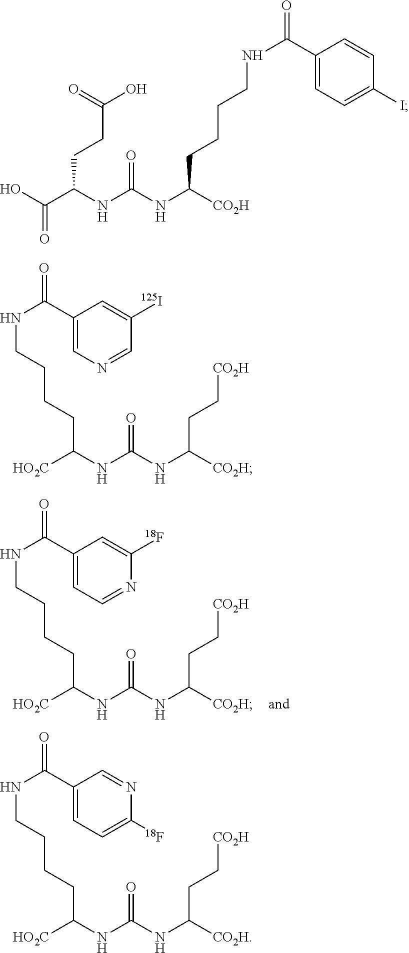

In some embodiments, the compound of formula (I) is selected from the group consisting of:

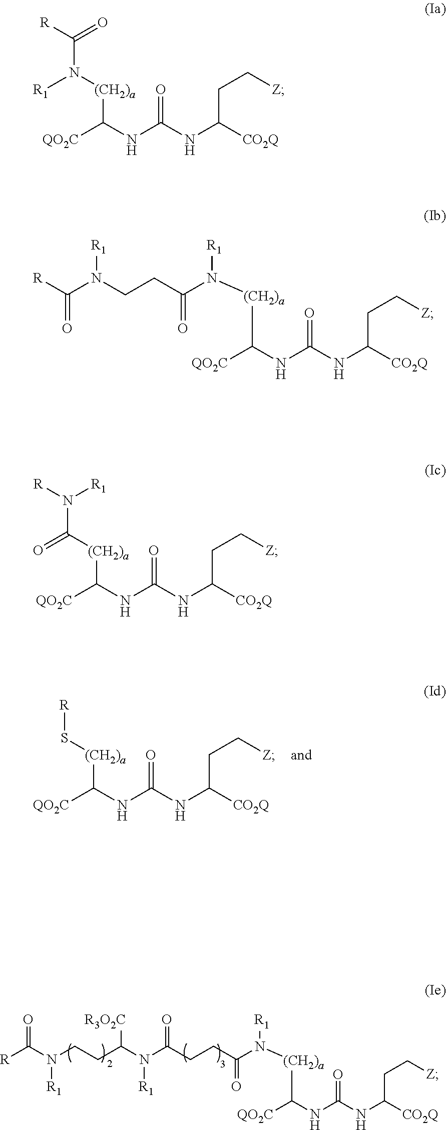

wherein Z, Q, R, R₁, R₃, a are defined as above; and stereoisomers and pharmaceutically acceptable salts thereof.

In other embodiments, the compound of formula (I) is selected from the group consisting of:

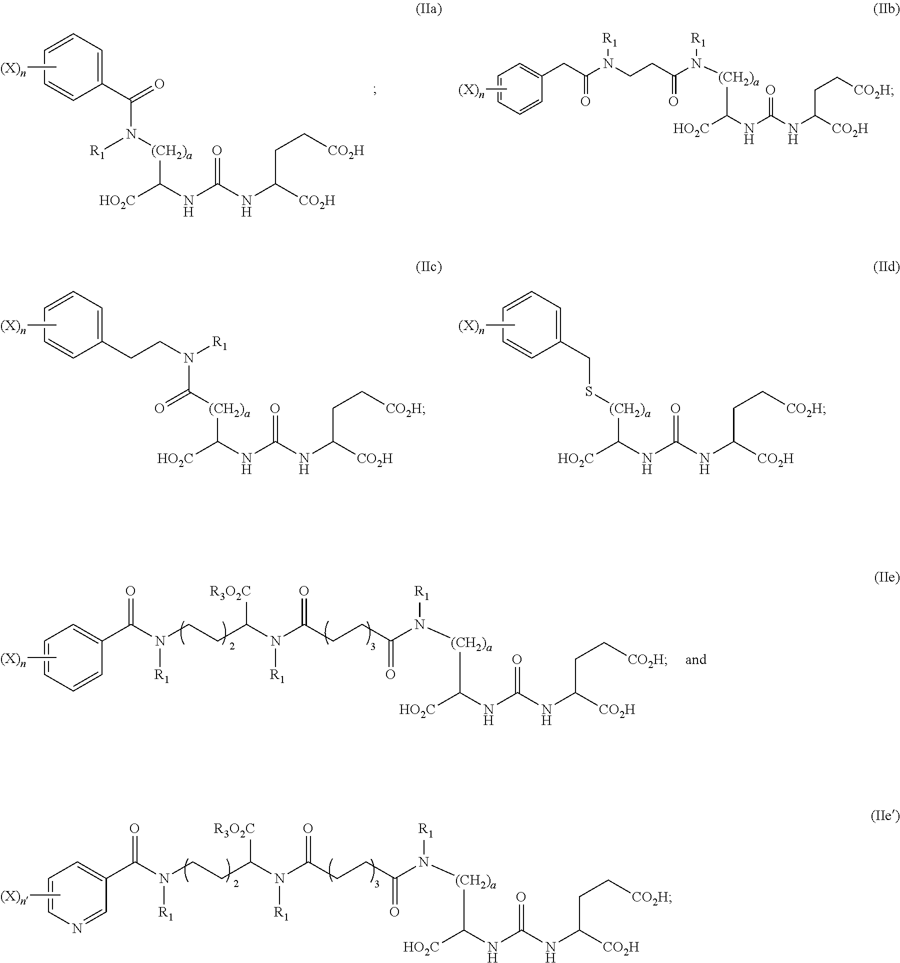

wherein Z, Q, R, R₁, R₃, X, a and n are defined hereinabove; and stereoisomers and pharmaceutically acceptable salts thereof. In particular embodiments, X is selected from the group consisting of ¹²⁵I, ¹²³I, ¹³¹I, ²¹¹At, ⁷⁷Br, and ^{80m}Br.

In particular embodiments, the compound of formula (I) is selected from the group consisting of:

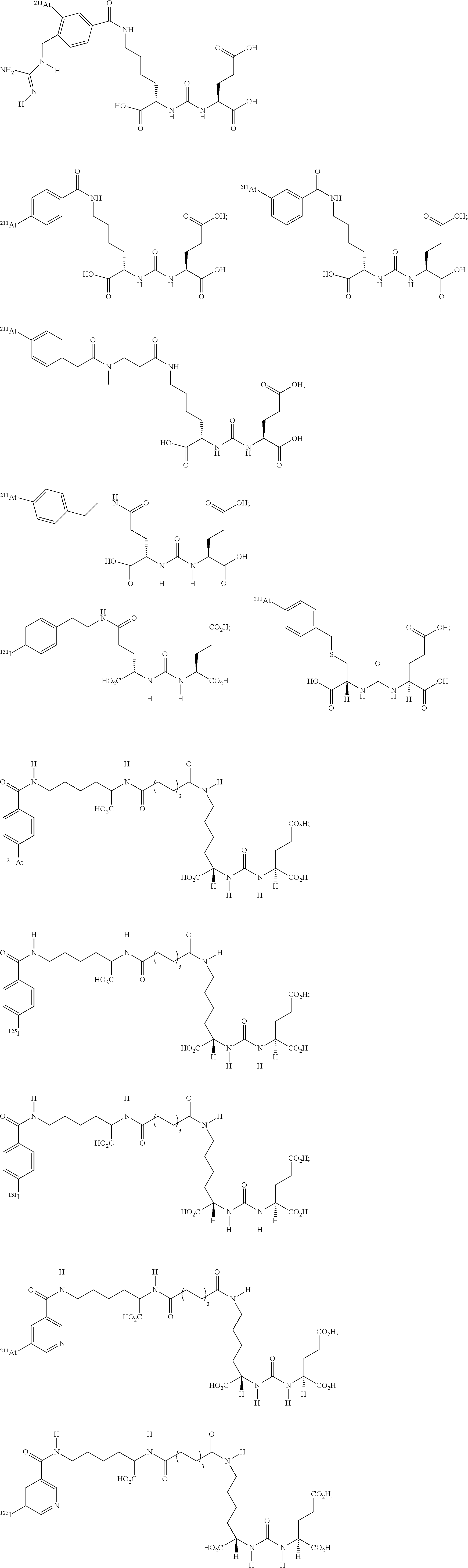

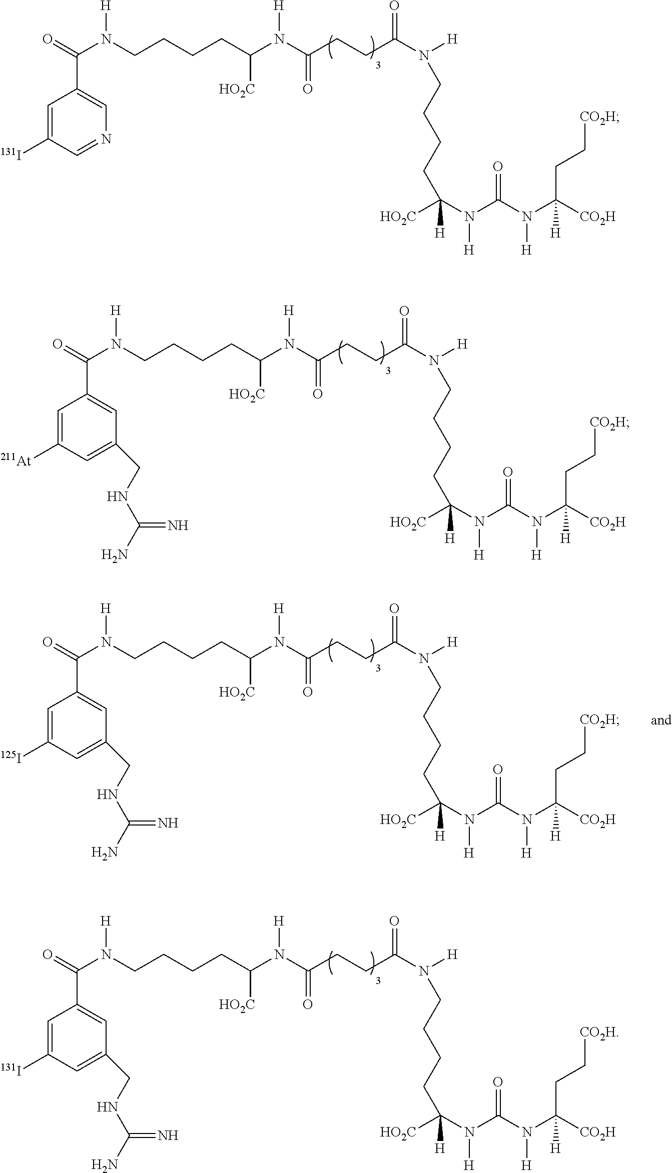

In yet more particular embodiments, the compound of formula (I) is selected from the group consisting of:

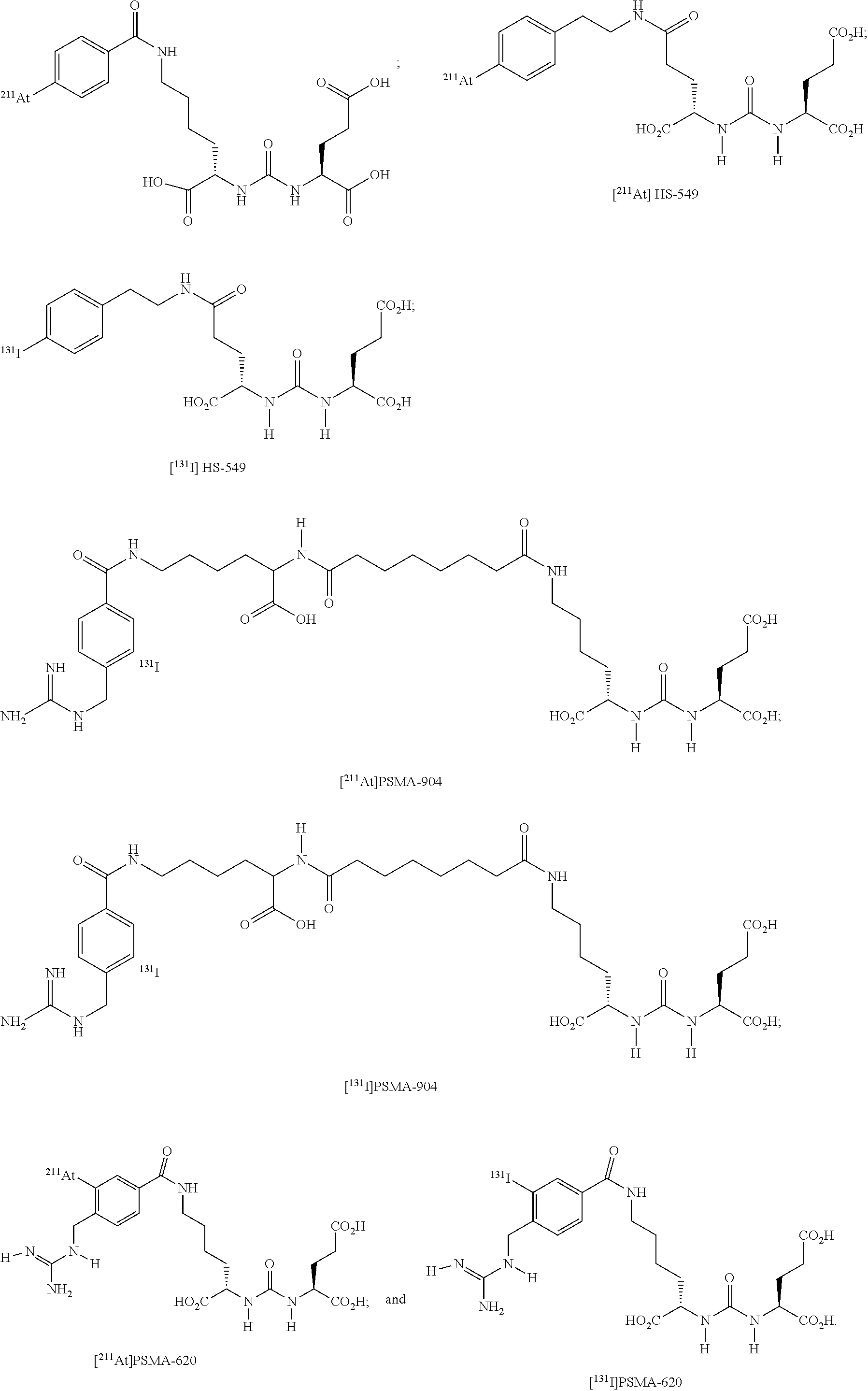

In other embodiments, the one or more PSMA-expressing tumor or cell is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof. In more specific embodiments, the one or more PSMA-expressing tumor or cell is a prostate tumor or cell. In some embodiments, the one or more PSMA-expressing tumors or cells are in vitro, in vivo, or ex vivo. In particular embodiments, the one or more PSMA-expressing tumors or cells are present in a subject.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In some embodiments, the compound of formula (I) is cleared from the subject's kidneys in about 24 hours.

In some embodiments, the presently disclosed methods use compounds that are stable in vivo such that substantially all, e.g., more than about 50%, 60%, 70%, 80%, or more preferably 90% of the injected compound is not metabolized by the body prior to excretion. In other embodiments, the compound comprising the imaging agent is stable in vivo.

In specific embodiments, the method results in inhibition of the tumor growth. As used herein, the term “inhibition” or “reduction” and grammatical derivations thereof, refers to the ability of an agent to block, partially block, interfere, decrease, reduce or deactivate a biological molecule, pathway or mechanism of action. Thus, one of ordinary skill in the art would appreciate that the term “inhibit” encompasses a complete and/or partial loss of activity, e.g., a loss in activity by at least 10%, in some embodiments, a loss in activity by at least 20%, 30%, 50%, 75%, 95%, 98%, and up to and including 100%.

In other specific embodiments, the compound of formula (I) completely occupies the binding cavity of the PSMA expressing tumors or cells.

### C. Pharmaceutical Composition Comprising Compounds of Formula (I)

In another aspect, the present disclosure provides a pharmaceutical composition including one compound of formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

Formula (I) does not include compounds disclosed in WO 2008/058192, WO 2010/014933, and U.S. Pat. No. 7,408,079, each of which is incorporated herein by reference in their entirety. More particularly, the following compounds are expressly disclaimed from the pharmaceutical composition claims in the present application:

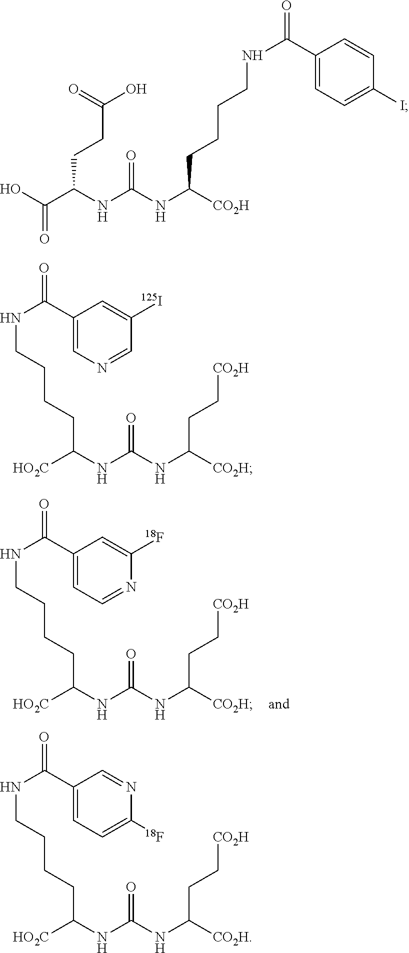

The term “combination” is used in its broadest sense and means that a subject is administered at least two agents, more particularly a compound of formula (I), including compounds of formula (Ia), (Ib), (Ic), (Id), (Ie), (Ha), (IIb), (IIc), (IId), (He), and (IIe′), and optionally, one or more therapeutic agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Advantageously, such combination therapies utilize lower dosages of the conventional therapeutics, thus avoiding possible toxicity and adverse side effects incurred when those agents are used as monotherapies.

The timing of administration of a compound of formula (I) including compounds of formula (Ia), (Ib), (Ic), (Id), (Ie), (IIa), (IIb), (IIc), (IId), (He), and (IIe′), and at least one additional therapeutic agent can be varied so long as the beneficial effects of the combination of these agents are achieved. Accordingly, the phrase “in combination with” refers to the administration of a compound of formula (I) including compounds of formula (Ia), (Ib), (Ic), (Id), (Ie), (IIa), (IIb), (IIc), (IId), (He), and (IIe′), and at least one additional therapeutic agent either simultaneously, sequentially, or a combination thereof. Therefore, a subject administered a combination of a compound of formula (I) including compounds of formula (Ia), (Ib), (Ic), (Id), (Ie), (IIa), (IIb), (IIc), (IId), (He), and (IIe′), and at least one additional therapeutic agent can receive compound of formula (I) including compounds of formula (Ia), (Ib), (Ic), (Id), (Ie), (Ha), (IIb), (IIc), (IId), (He), and (IIe′), and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of formula (I), including compounds of formula (Ia), (Ib), (Ic), (Id), (Ie), (Ha), (IIb), (IIc), (IId), (He), and (IIe′), and at least one additional therapeutic agent are administered simultaneously, they can be administered to the subject as separate pharmaceutical compositions, each comprising either a compound of formula (I), including compounds of formula (Ia), (Ib), (Ic), (Id), (Ie), (IIa), (IIb), (IIc), (IId), (He), and (IIe′), or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of formula (I), including compounds of formula (Ia), (Ib), (Ic), (Id), (Ie), (IIa), (IIb), (IIc), (IId), (He), and (IIe′), and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000).

In therapeutic and/or diagnostic applications, the compounds of the disclosure can be formulated for a variety of modes of administration, including systemic and localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries, which facilitate processing of the active compounds into preparations which can be used pharmaceutically.

### II. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of Formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Description of compounds of the present disclosure is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups that are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen molecule.

Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—), —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

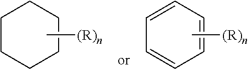

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

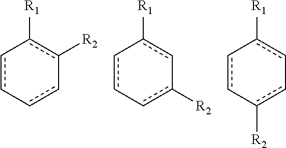

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′Rθ, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR′″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example iodine-125 (¹²⁵I) or astatine-211 (²¹¹At) All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)— catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

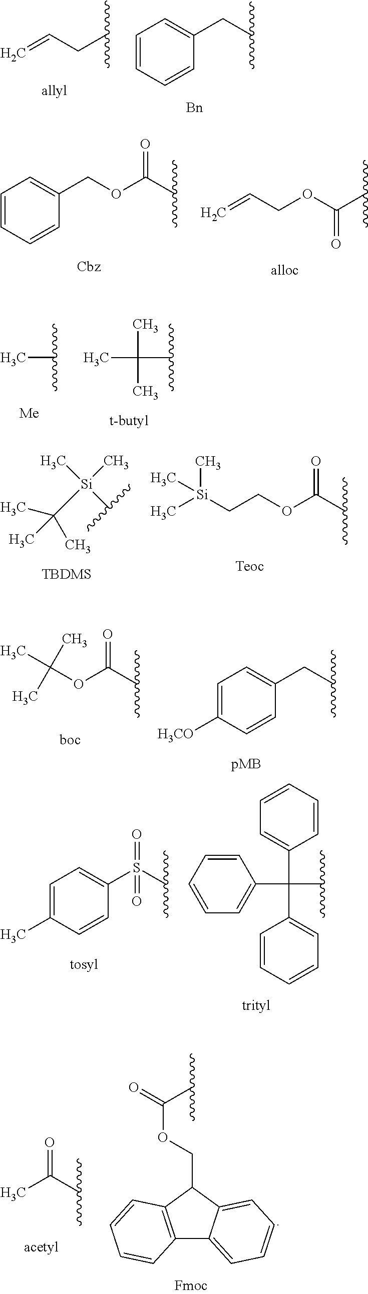

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

In the examples below the following terms are intended to have the following meaning: ACN: acetonitrile, DCM: Dichloromethane, DIPEA: N,N-Diisopropylethylamine, DMF: Dimethylformamide, HPLC: High Performance Liquid Chromatography, HRMS: High Resolution Mass Spectrometry, LRMS: Low Resolution Mass Spectrometry, NCS: N-Chlorosuccinimide, NHS: N-Hydroxysuccinimide, NMR: nuclear magnetic resonance, PMB: α-methoxybenzyl, RT: room temperature, TEA: Triethylamine, TFA: Trifluoroacetic acid, and TSTU: O-(N-Succinimidyl)-N,N,N′,N′-tetramethyluronium tetrafluoroborate.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Radiolabeled PSMA Binding Scaffolds for Targeted Radioimmunotherapy

The development of low molecular weight radiotherapeutic agents is much different from developing radiopharmaceuticals for imaging in that longer tumor residence times are required for the former. The compounds disclosed in the present patent application are based on extensive structure-activity relationships, not merely of the imaging precursors or PSMA binding compounds, but on actual imaging agents already synthesized and tested in vivo as well as on molecular modeling, including co-crystallization of several of existing agents with PSMA. Moreover, the PSMA becomes internalized upon binding of at least a subset of the inhibitors possibly adding further to a therapeutic effect.

Many radionuclides, primarily β- and alpha emitters, have been investigated for targeted radioimmunotherapy, including radiohalogens and radiometals (Table 1).

TABLE 1
Representative Therapeutic Radionuclides

| | |
|---|---|
| β-particle emitters | ⁹⁰Y, ¹³¹I, ¹⁷⁷Lu, ¹⁵³Sm, ¹⁸⁶Re, |
| | ¹⁸⁸Re, ⁶⁷Cu, ²¹²Pb, ¹⁶⁶Ho, ⁴⁷Sc |
| α-particle emitters | ²²⁵Ac, ²¹³Bi, ²¹²Bi, ²¹¹At, ²¹²Pb, |
| | ²²⁷Th, ²²³Ra |
| Auger electron emitters | ¹²⁵I, ¹²³I, ⁶⁷Ga, ¹¹¹In, ⁷⁷Br, ^{80m}Br |

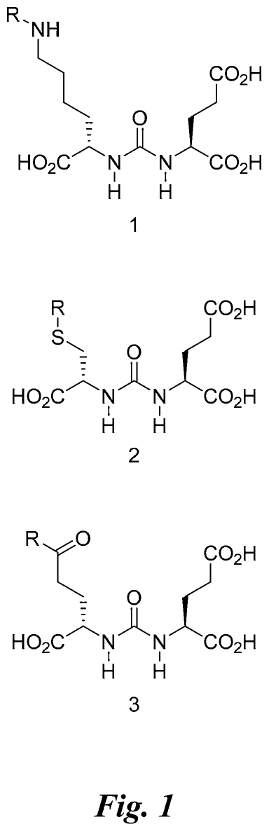

The presently disclosed subject matter, in some embodiments, includes radiohalogens ¹²⁵I, ¹²³I, ¹³¹I, ²¹¹At, ⁷⁷Br, and ^{80m}Br, with several specific examples of appropriate ways of introducing them into PSMA-targeting molecules. These radiohalogens are covalently bound to the targeting moiety and unlike large chelated radiometals are small enough that the entire radiolabeled PSMA inhibitor can fit within the PSMA binding cavity thereby retaining the high binding affinity. The same radiolabeled prosthetic groups can be conjugated to linker-inhibitor urea conjugates to move the radiolabeled portion of the inhibitor to the exterior of the protein while the glutamate and urea moieties remain in the binding cavity. Radiohalogenated PSMA binding radiotherapeutics can be built upon three PSMA binding scaffolds: lysine-glutamate urea 1, cysteine-glutamate urea 2, and glutamate-glutamate urea 3 (FIG. 1), as outlined in Example 2.

### Example 2

### Synthesis and Radiochemistry

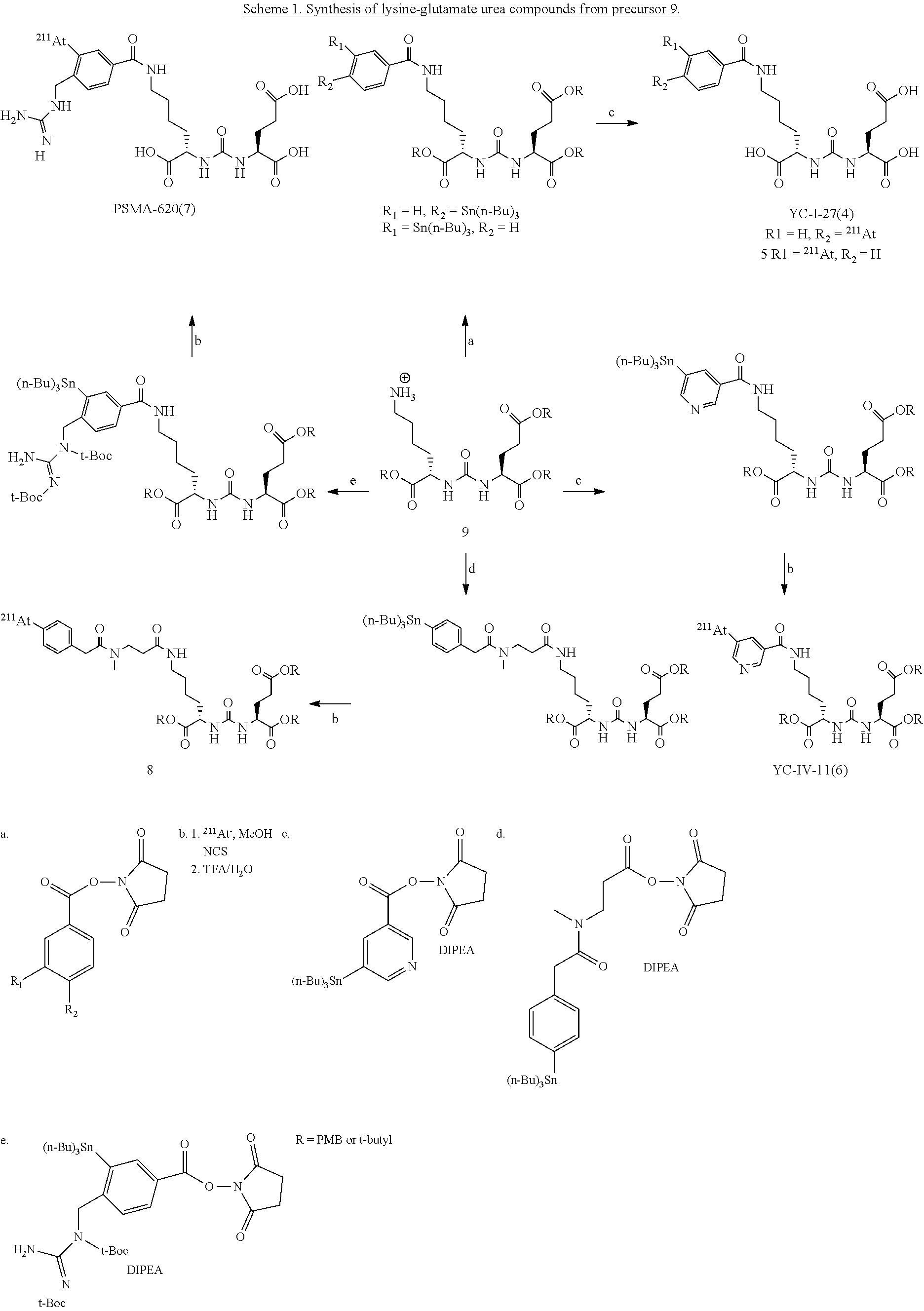

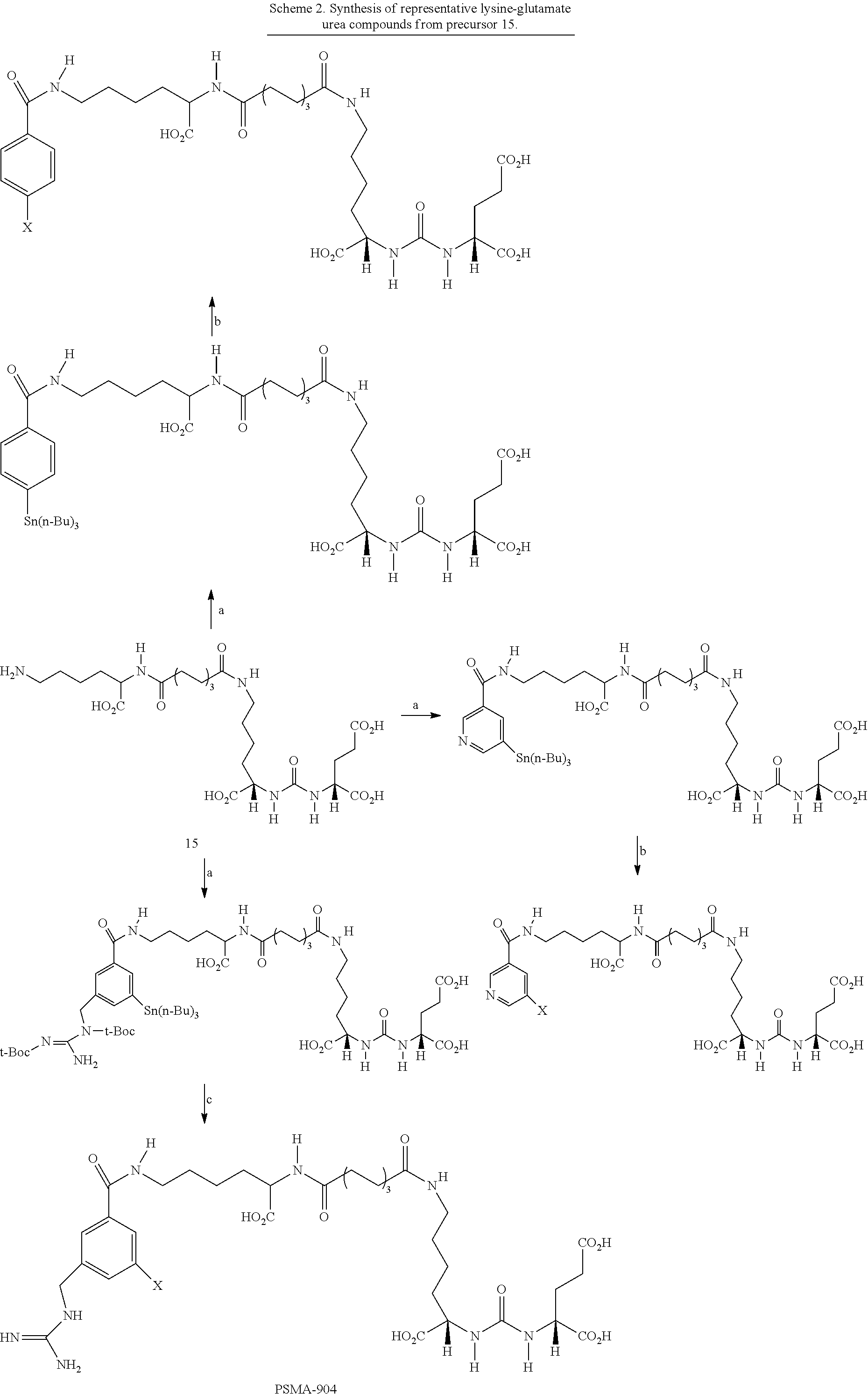

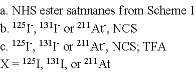

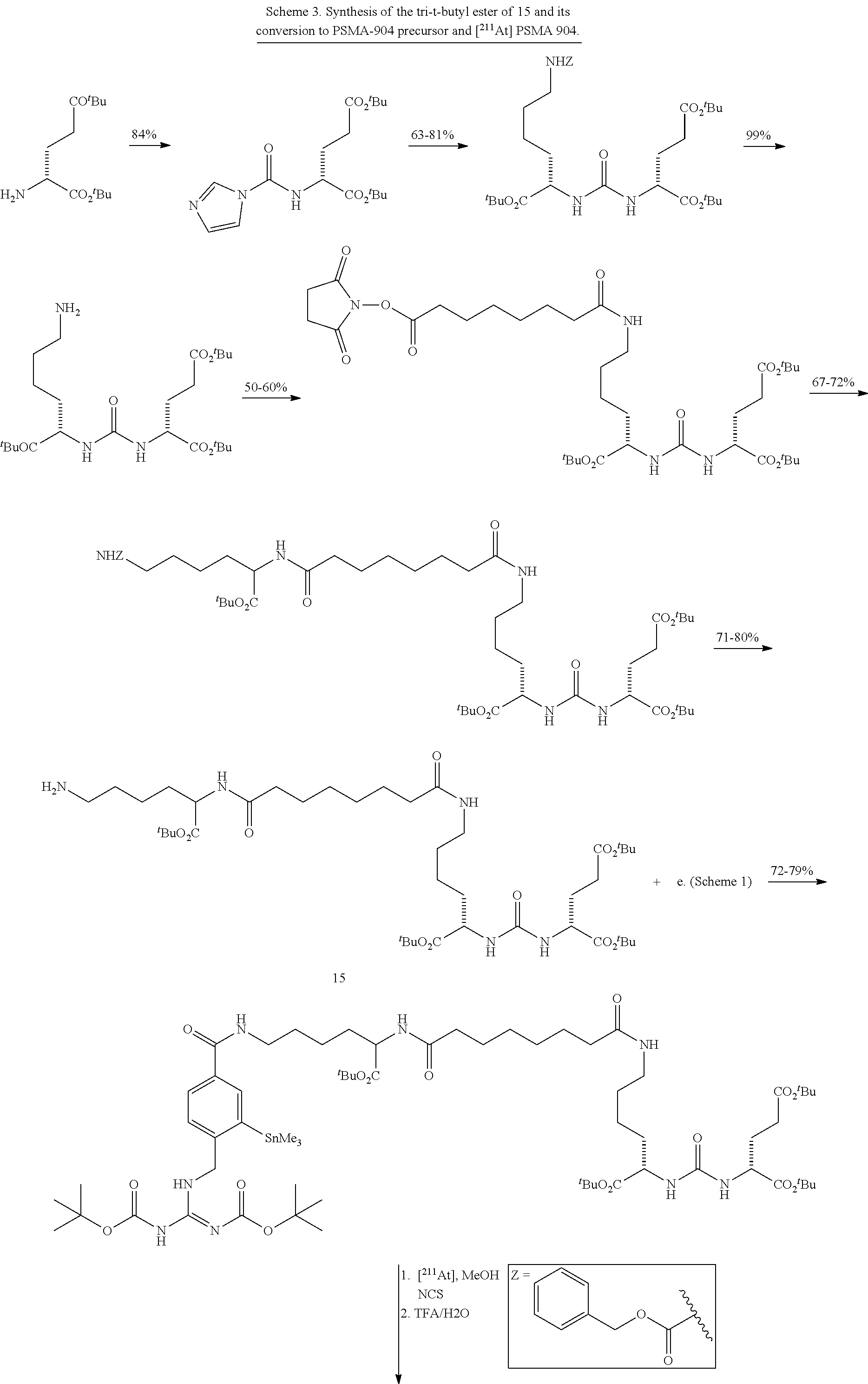

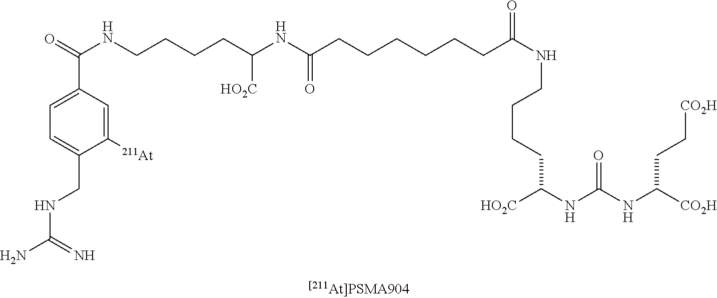

Another synthetic route for [²¹¹At]PSMA 904 precursor is outlined in detail below (Schemes 4 to 7).

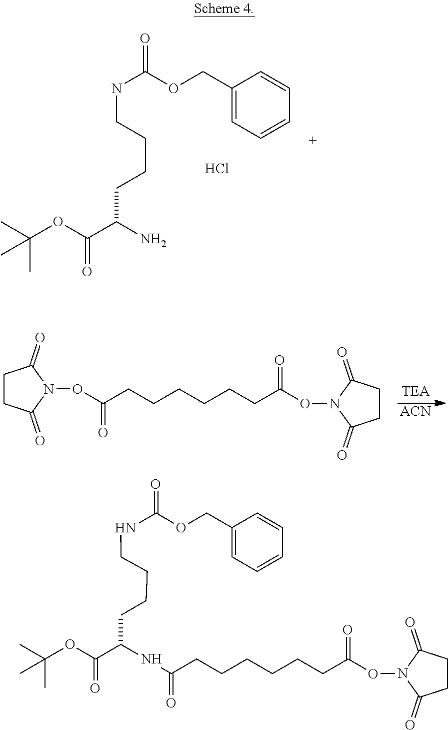

Synthesis of (S)-2,5-dioxopyrrolidin-1-yl 8-((6-(((benzyloxy)carbonyl)amino)-1-(tert-butoxy)-1-oxohexan-2-yl)amino)-8-oxooctanoate. Referring to Scheme 4, a solution of (S)-tert-butyl 2-amino-6-(((benzyloxy)carbonyl)amino) hexanoate hydrochloride (1.01 g, 2.71 mmol) in acetonitrile (50 mL) was added over a period of 30 min to a solution of bis (2,5-dioxopyrrolidin-1-yl) octanedioate (1.0 g, 2.71 mmol) and triethylamine (0.38 mL, 2.71 mmol) in acetonitrile (50 mL) and the mixture stirred at 20° C. for 4 h. Acetonitrile was evaporated to reduce the volume to half and the remaining mixture was partitioned between water and ethyl acetate. Pooled ethyl acetate solution was dried with anhydrous sodium sulfate and concentrated. The crude product was purified using a Biotage 25 g SNAP ULTRA column and 7:3 hexanes:ethyl acetate as mobile phase to yield 1.0 g (1.70 mmol; 62.5%) of (S)-2,5-dioxopyrrolidin-1-yl 8-((6-(((benzyloxy)carbonyl)amino)-1-(tert-butoxy)-1-oxohexan-2-yl)amino)-8-oxooctanoate as a white solid: ¹H-NMR: δ_{H} (400 MHz, C²HCl₃) 7.38-7.25 (5H, m), 6.17-6.08 (1H, d), 5.06 (3H, m), 4.95-4.87 (1H, bs), 4.50-4.42 (1H, m), 3.38-3.30 (1H, m), 3.32-3.04 (4H, m), 2.86-2.72 (6H, m), 2.62-2.52 (3H, m), 2.22-2.18 (2H, t), 1.81-1.06 (16H, m).

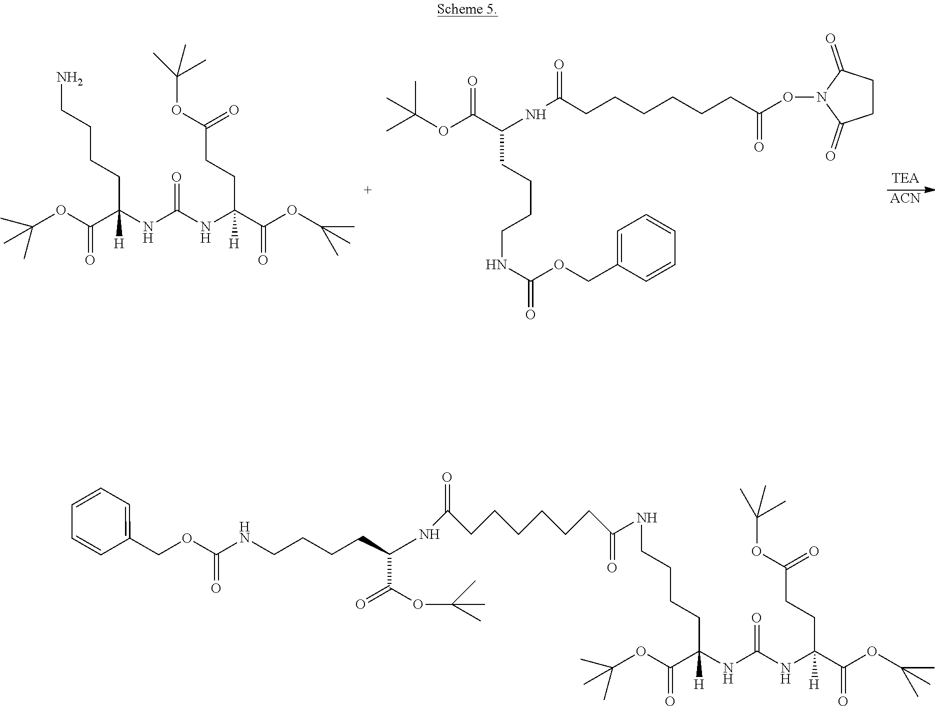

Synthesis of (24S,28S)-tetra-tert-butyl 3,11,18,26-tetraoxo-1-phenyl-2-oxa-4,10,19,25,27-pentaazatriacontane-9,24,28,30-tetracarboxylate. Referring now to Scheme 5, triethylamine (0.17 mL, 1.23 mmol) was added to a solution of (S)-di-tert-butyl 2-(3-((S)-6-amino-1-(tert-butoxy)-1-oxohexan-2-yl)ureido)pentanedioate (0.50 g, 1.03 mmol) and (S)-2,5-dioxopyrrolidin-1-yl 8-((6-(((benzyloxy)carbonyl)amino)-1-(tert-butoxy)-1-oxohexan-2-yl)amino)-8-oxooctanoate (0.61 g, 1.03 mmol) in acetonitrile (40 mL) kept at 5-10° C. After two hours, the reaction mixture was partitioned between water and ethyl acetate. The pooled ethyl acetate solution was dried with anhydrous magnesium sulfate and concentrated. The residue was purified using a Biotage 25 g SNAP ULTRA column and 5:1 hexanes:ethyl acetate as mobile phase to yield 0.77 g (0.80 mmol; 78%) of (24S,28S)-tetra-tert-butyl 3,11,18,26-tetraoxo-1-phenyl-2-oxa-4,10,19,25,27-pentaazatriacontane-9,24,28,30-tetracarboxylate as a white foam.

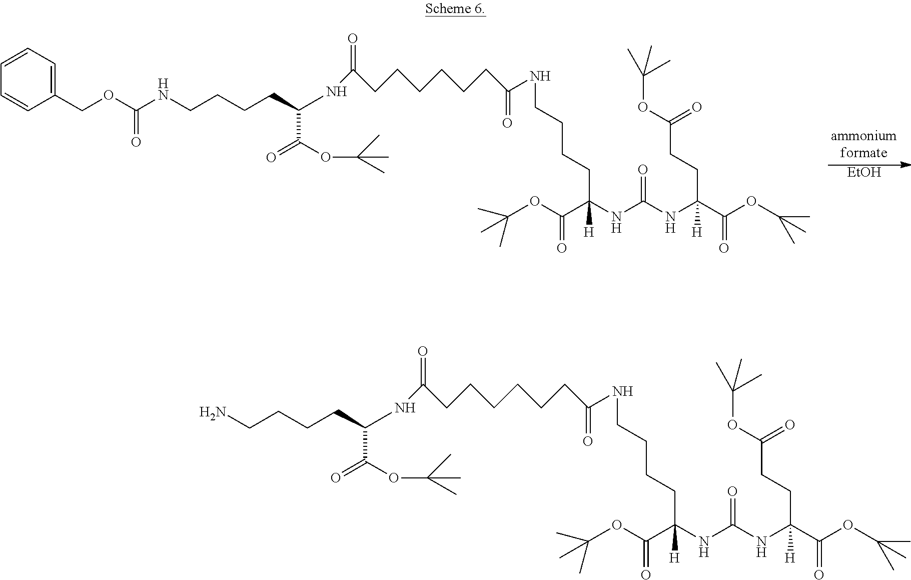

Synthesis of (3S,7S)-tetra-tert-butyl 26-amino-5,13,20-trioxo-4,6,12,21-tetraazahexacosane-1,3,7,22-tetracarboxylate. Referring now to Scheme 6, ammonium formate (0.46 g, 7.27 mmol) and (24S, 28S)-tetra-tert-butyl 3,11,18,26-tetraoxo-1-phenyl-2-oxa-4,10,19,25,27-pentaazatriacontane-9,24,28,30-tetracarboxylate (0.70 g, 0.73 mmol) were dissolved in ethanol (25 mL) and the solution was degassed. Palladium on carbon (0.10 g, 0.94 mmol) was added and the solution was degassed again. Hydrogen was introduced to the flask and the mixture was stirred under a hydrogen atmosphere for 16 h. The solution was degassed and the flask was purged with argon. The mixture was filtered over Celite-545 bed, and the bed was washed with 50 mL of ethanol. The filtrate was concentrated to dryness, and the residue was taken in DCM and filtered through Celite to remove any insolubles. The filtrate was concentrated to dryness to give 0.47 g (0.57 mmol, 78% yield) of (3S,7S)-tetra-tert-butyl 26-amino-5,13,20-trioxo-4,6,12,21-tetraazahexacosane-1,3,7,22-tetracarboxylate.

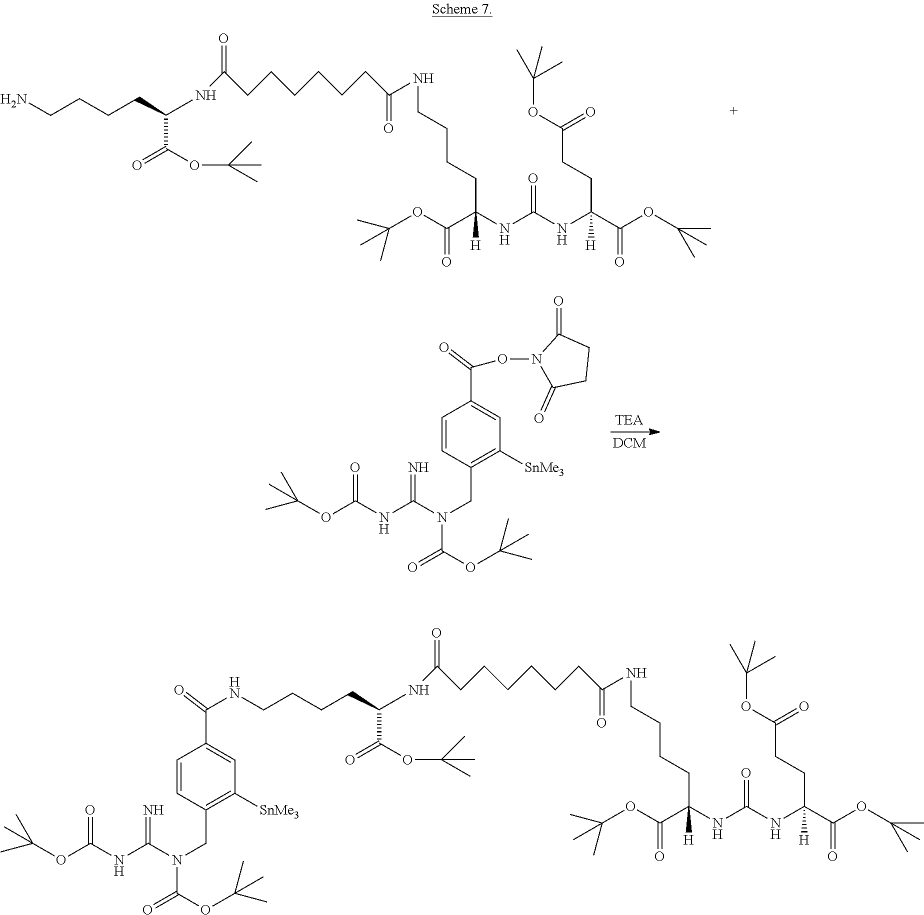

Synthesis of (22S,26S)-tetra-tert-butyl 1-(4-((1,3-bis(tert-butoxycarbonyl)guanidino)methyl)-3-(trimethylstannyl)phenyl)-1,9,16,24-tetraoxo-2,8,17,23,25-pentaazaoctacosane-7,22,26,28-tetracarboxylate ([²¹¹At]PSMA 904 precursor). Referring now to Scheme 7, TEA (44.0 mg, 0.23 mmol) was added to a solution of (3S,7S)-tetra-tert-butyl 26-amino-5,13,20-trioxo-4,6,12,21-tetraazahexacosane-1,3,7,22-tetracarboxylate (57.0 mg, 0.07 mmol) and 2,5-dioxopyrrolidin-1-yl 4-((1,3-bis(tert-butoxycarbonyl)guanidino)methyl)-3-(trimethylstannyl)benzoate (30.0 mg, 0.05 mmol) in dichloromethane (15 mL) cooled to 5-10° C. The mixture was stirred at 20° C. for 16 h and 15 mL water was added to that. The pooled dichloromethane layer was dried with anhydrous sodium sulfate and dichloromethane evaporated. The crude material was subjected preparative thin layer chromatography using 1:1 hexane:ethyl acetate to yield 40 mg (0.03 mmol, 63.8% yield) of (22S,26S)-tetra-tert-butyl 1-(4-((1,3-bis(tert-butoxycarbonyl)guanidino)methyl)-3-(trimethylstannyl)phenyl)-1,9,16,24-tetraoxo-2,8,17,23,25-pentanzaoctacosane-7,22,26,28-tetracarboxylate as a tan solid: ¹HMR δ_{H} (400 MHz, C²HCl₃) 9.50-9.25 (2H, bs), 7.88 (1H, s), 7.61-7.65 (1H, d), 6.97-6.93 (1H, d), 6.70-6.65 (1H, t), 6.42-6.33 (2H, m), 5.66-5.6 (1H, m), 5.55-5.00 (1H, m), 5.22 (2H, s), 4.52-4.45 (1H, m), 4.33-4.23 (2H, m), 3.45-3.31 (2H, m), 3.28-3.18 (1H, m), 3.13-3.05 (1H, m), 2.32-1.08 (82H, m), 0.36 (9H, s). LRMS: Cluster peaks at 1367.8 (M+H)⁺. HRMS: Calcd for C₆₄H₁₁₁N₈O₁₆Sn (M+H)⁺: 1367.7140; Found: 1367.7147±0.0007 (n=4).

The synthesis of PSMA-904 is disclosed in Schemes 8 and 9 below.

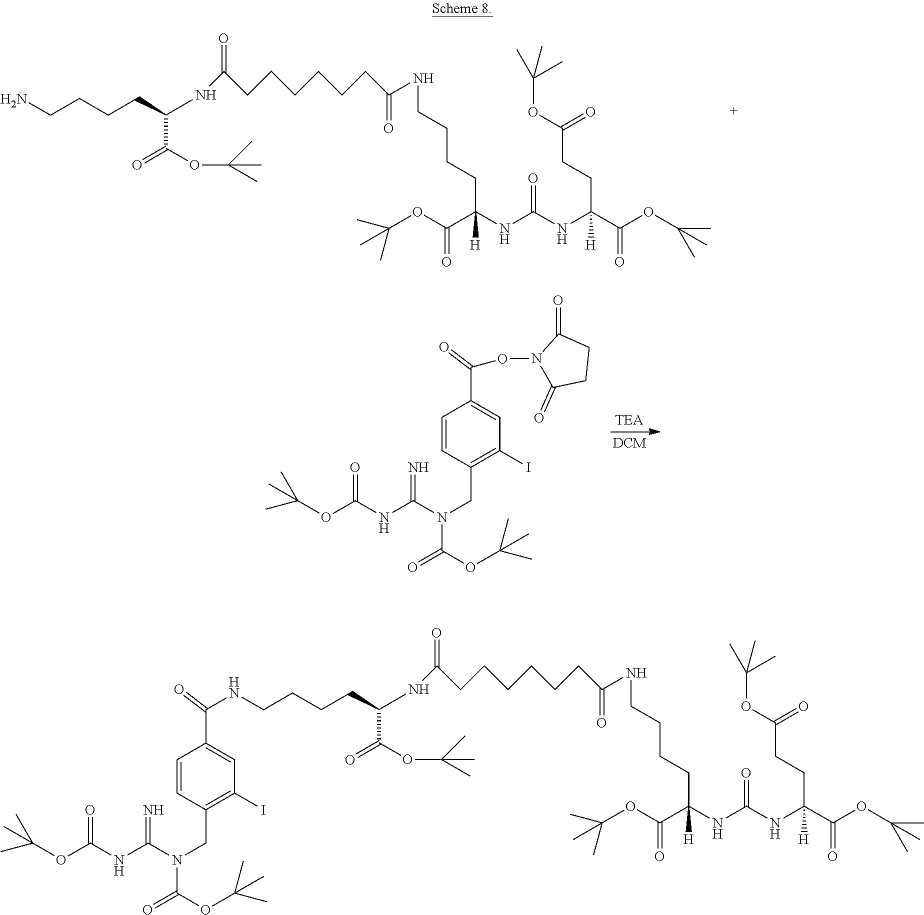

Synthesis of (22S,26S)-tetra-tert-butyl 1-(4-((1,3-bis(tert-butoxycarbonyl)guanidino)methyl)-3-iodophenyl)-1,9,16,24-tetraoxo-2,8,17,23,25-pentaazaoctacosane-7,22,26,28-tetracarboxylate. Referring now to Scheme 8, triethylamine (38.9 mg, 0.20 mmol) was added to a solution of (3S,7S)-tetra-tert-butyl 26-amino-5,13,20-trioxo-4,6,12,21-tetraazahexacosane-1,3,7,22-tetracarboxylate (50.4 mg, 0.06 mmol) and 2,5-dioxopyrrolidin-1-yl 4-((1,3-bis(tert-butoxycarbonyl)guanidino)methyl)-3-iodobenzoate (25.00 mg, 0.04 mmol) in 15 mL of dichloromethane kept at 5-10° C. The reaction was allowed to proceed at 20° C. for 16 h and 15 mL water was added. The organic layer was separated, dried with anhydrous sodium sulfate, and dichloromethane was evaporated. The crude material was subjected to thick layer chromatography using hexanes and ethyl acetate (1:1) to give 30 mg (0.023 mmol, 55.6% yield) of (22S,26S)-tetra-tert-butyl 1444(1,3-bis(tert-butoxycarbonyl)guanidino)methyl)-3-iodophenyl)-1,9,16,24-tetraoxo-2,8,17,23,25-pentanzaoctacosane-7,22,26,28-tetracarboxylate as a tan solid: ¹H-NMR δ_{H} (400 MHz, C²HCl₃) 9.58-9.35 (2H, bs), 8.36 (1H, s), 7.84-7.80 (2H, d), 7.10-6.98 (2H, d), 6.46-6.30 (2H, m), 5.70-5.34 (2H, m), 5.20 (2H, s), 4.58-4.52 (1H, m), 4.38-4.28 (2H, m), 3.52-3.07 (4H, m), 2.36-2.05 (8H, m), 1.92-1.27 (73H, m). LRMS: 1329.6 (M+H)⁺, 1351.6 (M+Na)⁺. HRMS: Calcd for C₆₁H₁₀₂IN₈O₁₆ (M+H)⁺: 1329.6458; Found: 1329.6456±0.0015 (n=4).

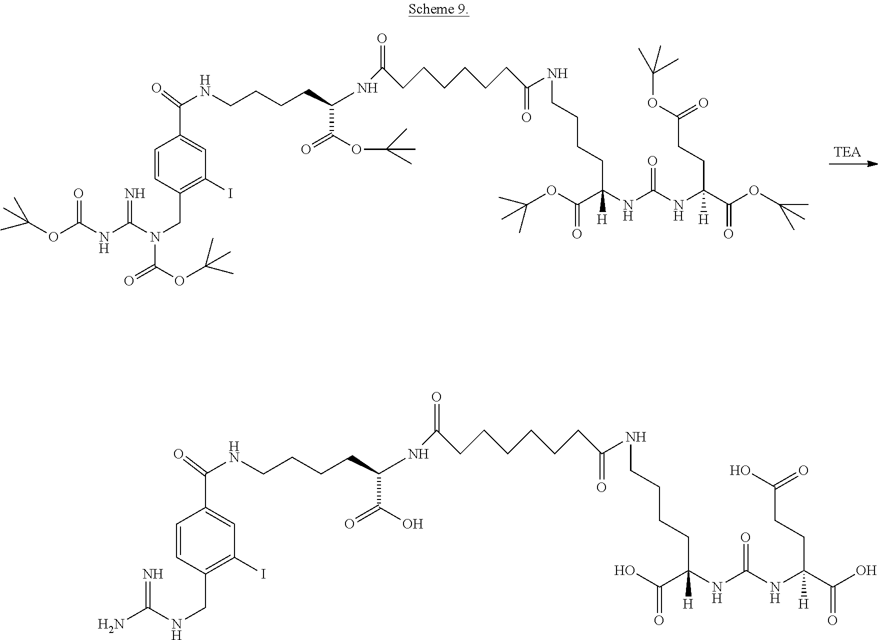

Synthesis of (22S,26S)-1-(4-(guanidinomethyl)-3-iodophenyl)-1,9,16,24-tetraoxo-2,8,17,23,25-pentaazaoctacosane-7,22,26,28-tetracarboxylic acid. Referring now to Scheme 9, trifluoroacetic acid (5.0 mL, 64.9 mmol) was added to (22S,26S)-tetra-tert-butyl 1-(4-((1,3-bis(tert-butoxycarbonyl)guanidino)methyl)-3-iodophenyl)-1,9,16,24-tetraoxo-2,8,17,23,25-pentaazaoctacosane-7,22,26,28-tetracarboxylate (56.0 mg, 0.04 mmol). The mixture was stirred at 20° C. for 17 h and trifluoroacetic was evaporated and the residue dried to obtain 40 mg (0.04 mmol of TFA salt, 94% yield) of (22S,26S)-1-(4-(guanidinomethyl)-3-iodophenyl)-1,9,16,24-tetraoxo-2,8,17,23,25-pentaazaoctacosane-7,22,26,28-tetracarboxylic acid of an oil. The crude oil was purified by reversed-phase HPLC LRMS: 905.3 (M+H)⁺. HRMS: Calcd for C₃₅H₅₄IN₈O₁₂ (M+H)⁺: 905.2906; Found: 905.2897±0.0006 (n=4).

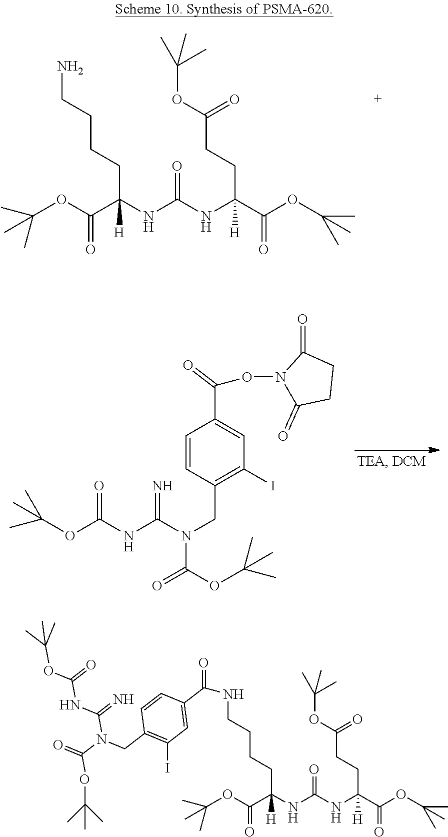

Synthesis of (S)-di-tert-butyl 2-(3-((S)-6-(4-((1,3-bis(tert-butoxycarbonyl)guanidino)methyl)-3-iodobenzamido)-1-(tert-butoxy)-1-oxohexan-2-yl)ureido)pentanedioate (PSMA-620). Referring now to Scheme 10, triethylamine (39.3 mg, 0.21 mmol) was added to a solution of (S)-di-tert-butyl 2-(3-((S)-6-amino-1-(tert-butoxy)-1-oxohexan-2-yl)ureido)pentanedioate (20.00 mg, 0.04 mmol) and 2,5-dioxopyrrolidin-1-yl 4-((1,3-bis(tert-butoxycarbonyl)guanidino)methyl)-3-iodobenzoate (25.3 mg, 0.04 mmol) in 20 mL of dichloromethane kept at 5-10° C. The reaction was allowed to proceed at 20° C. for 16 h and 20 mL water was added. The organic layer was separated, dried with anhydrous sodium sulfate, and dichloromethane was evaporated. The crude material was subjected to preparative thick layer chromatography using 1:1 hexanes:ethyl acetate to obtain 28 mg (0.03 mmol, 69.0% yield) of (S)-di-tert-butyl 2-(3-((S)-6-(4-41,3-bis(tert-butoxycarbonyl)guanidino)methyl)-3-iodobenzamido)-1-(tert-butoxy)-1-oxohexan-2-yl)ureido)pentanedioate (28 mg, 0.028 mmol, 69.0% yield) as an off white solid: δ_{H} (400 MHz, C²HCl₃) 9.50-9.30 (2H, bs), 8.28 (1H, s), 7.80 (1H, d), 7.00 (1H, d), 6.85-6.70 (1H, m), 5.25-5.18 (3H, m), 4.37-4.27 (2H, m), 3.47-3.33 (2H, m), 2.47-2.00 (3H, m), 2.40-1.22 (53H, m). HRMS: Calcd for C₄₃H₇₀IN₆O₁₂ (M+H)⁺: 989.4096; Found: 989.4082±0.0005 (n=4).

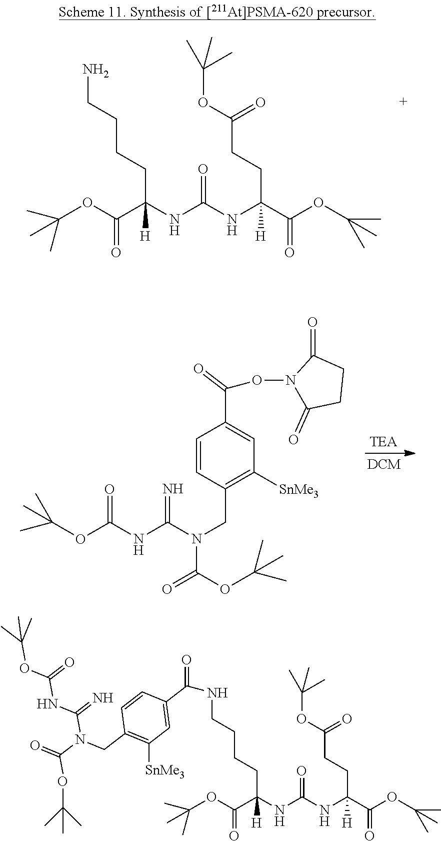

Synthesis of (S)-di-tert-butyl 2-(3-((S)-6-(4-((1,3-bis(tert-butoxycarbonyl)guanidino)methyl)-3-(trimethylstannyl)benzamido)-1-(tert-butoxy)-1-oxohexan-2-yl)ureido)pentanedioate ([²¹¹At]PSMA-620 precursor). Referring now to Scheme 11, triethylamine (39.3 mg, 0.21 mmol) was added to a solution of (S)-di-tert-butyl 2-(3-((S)-6-amino-1-(tert-butoxy)-1-oxohexan-2-yl)ureido)pentanedioate (20.00 mg, 0.04 mmol) and 2,5-dioxopyrrolidin-1-yl bis(tert-butoxycarbonyl)guanidino)methyl)-3-(trimethylstannyl)benzoate (26.8 mg, 0.04 mmol) in 20 mL of dichloromethane kept at 5-10° C. The reaction was allowed to proceed at 20° C. for 16 h and 15 mL water was added. The organic layer was separated, dried with anhydrous sodium sulfate, and dichloromethane was evaporated. The crude material was subjected to preparative thick layer chromatography using 1:1 hexanes:ethyl acetate to obtain 30 mg (0.03 mmol, 71.3% yield) of (S)-di-tert-butyl 2-(3-((S)-6-(4-((1,3-bis(tert-butoxycarbonyl)guanidino)methyl)-3-(trimethylstannyl)benzamido)-1-(tert-butoxy)-1-oxohexan-2-yl)ureido)pentanedioate as a foam: ¹H-NMR δ_{H} (400 MHz, C²HCl₃) 9.56-9.30 (2H, bs), 7.80 (1H, s), 7.70-7.65 (1H, d), 7.03-6.99 (1H, d), 6.55-6.50 (1H, dd), 5.27-5.18 (4H, m), 4.37-4.28 (2H, m), 3.47-3.40 (2H, m), 2.47-2.00 (3H, m), 1.90-1.10 (52H, m), 0.10 (9H, s). HRMS: Calcd for C₄₆H₇₉N₆O₁₂Sn (M+H)⁺: 1027.4778; Found: 1027.4784±0.0004 (n=4).

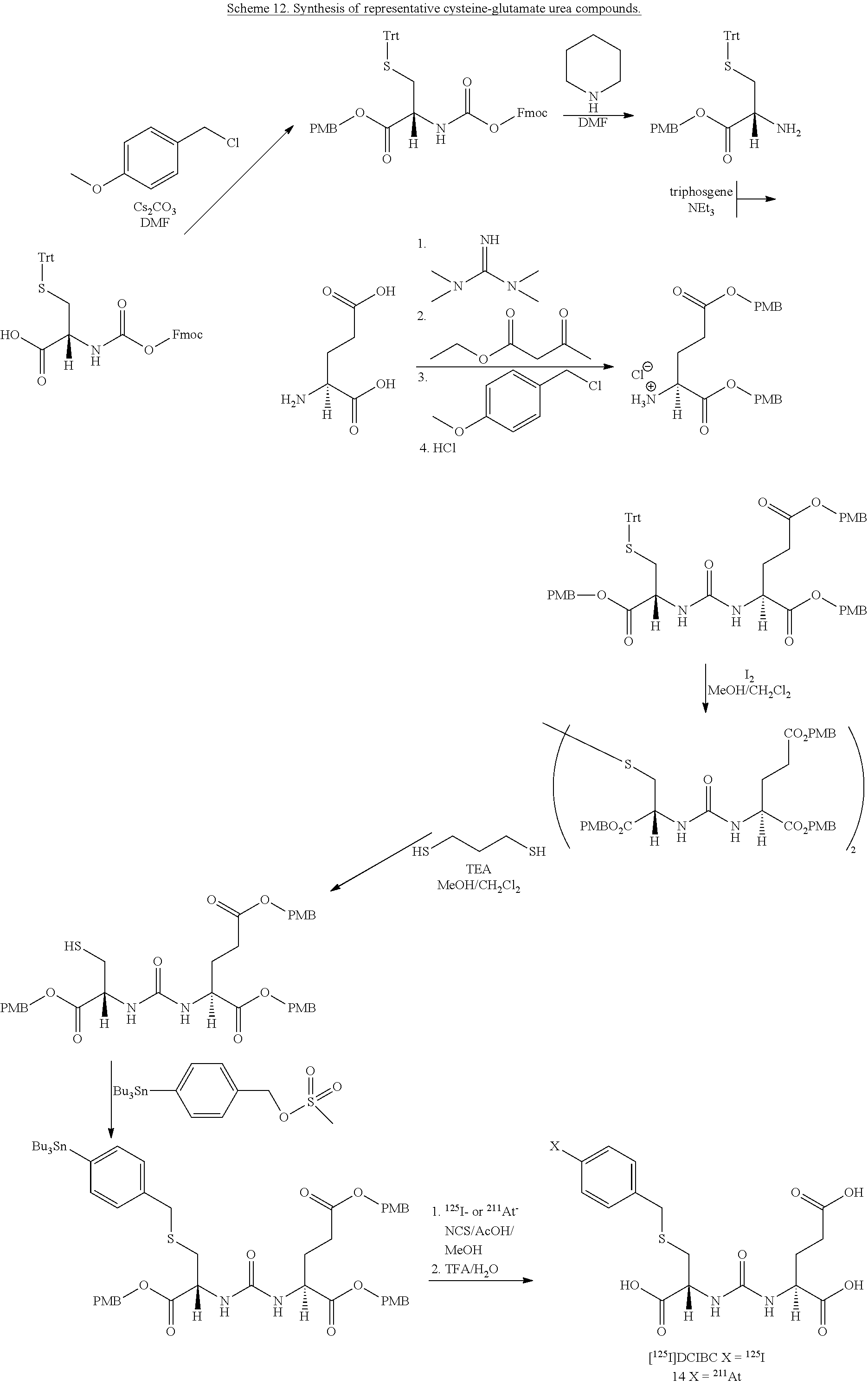

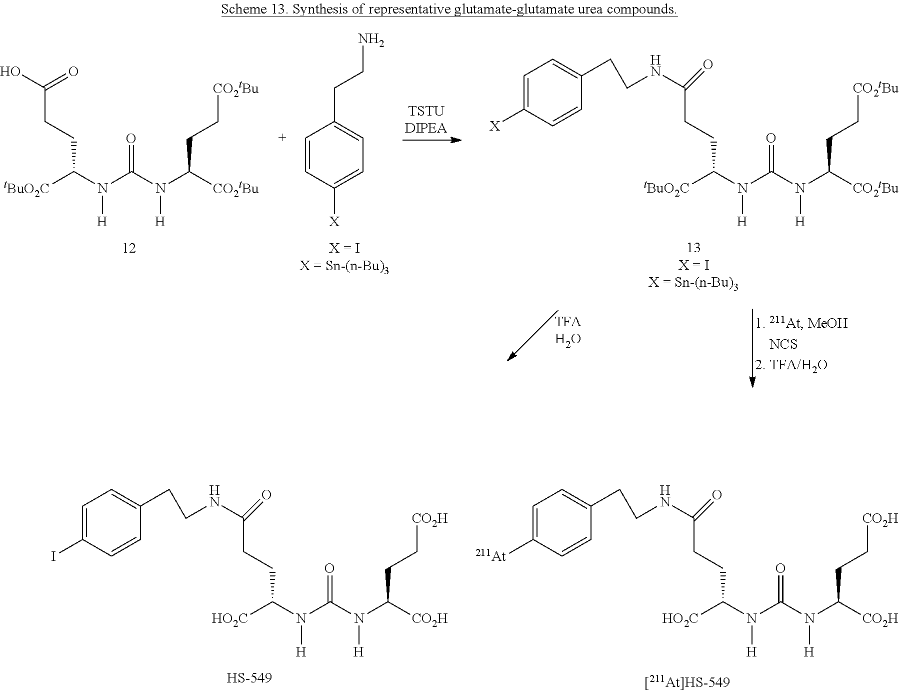

Synthesis of Di-tert-butyl (((S)-1-(tert-butoxy)-5-((4-iodophenethyl)amino)-1,5-dioxopentan-2-yl)carbamoyl)-L-glutamate. Referring now to Scheme 13, a mixture of 12 (0.100 g, 0.20 mmol) (Kularatne et al., 2009), TSTU (0.068 g, 0.22 mmol) and DIPEA (0.053 g, 0.41 mmol) were stirred in DMF (1 mL) at RT for 5 h. p-Iodophenethlamine (0.058 g, 0.20 mmol) was added dropwise after dilution with DMF (1 mL). The reaction mixture was stirred overnight, concentrated and purified by C-18 column chromatography eluting with 70-100% acetonitrile/H₂O provided 0.140 g (95%) of oily material. ¹H NMR (500 MHz, CDCl₃) δ ppm 7.57 (d, J=10 Hz, 2H), 7.37 (bs, 1H), 6.94 (d, J=10 Hz, 2H), 5.89-5.80 (m, 2H), 4.27-4.18 (m, 2H), 3.49-3.40 (m, 2H), 2.75 (t, J=5 Hz, 2H), 2.39-2.21 (m, 2H), 2.07-2.05 (m, 2H), 1.87-1.75 (m, 2H), 1.42 (m, 27H); ESMS m/z: 718.2 (M+H)⁺.

Synthesis of (45)-1-Carboxy-4-((4-iodophenethyl)amino)-4-oxobutyl)carbamoyl)-L-glutamic acid (HS 549). Referring to Scheme 13, a cold solution of 50% TFA/CH₂C₁₂ (2 mL) was added to Di-tert-butyl (((S)-1-(tert-butoxy)-5-((4-iodophenethyl)amino)-1,5-dioxopentan-2-yl)carbamoyl)-L-glutamate (0.140 g, 0.19 mmol) and stirred at RT for 2 h. The reaction mixture was concentrated and purified by C-18 column chromatography eluting with 70-90% MeOH/water, to provide 0.067 g (62%) of yellowish semisolid product, which was lyophilized. ¹H NMR (500 MHz, CD₃CN+D₂O (1:1)) δ ppm 8.11 (d, J=5 Hz, 2H), 7.50 (d, J=5 Hz, 2H), 4.68-4.67 (m, 1H), 4.61-4.59 (m, 1H), 3.82-3.79 (m, 2H), 3.19-3.16 (m, 2H), 2.88 (t, J=5 Hz, 2H), 2.68-2.65 (m, 2H), 2.57-2.54 (m, 1H), 2.46 (m, 2H), 2.37-2.27 (m, 2H); ¹³C NMR (125 MHz, CD₃CN+D₂O (1:1)) δ ppm 177.0, 176.3, 174.9, 159.5, 140.2, 138.4, 132.1, 119.5, 91.9, 53.5, 53.3, 41.2, 35.2, 33.0, 30.9, 28.7, 27.6. ESMS m/z: 550.0 (M+H)⁺.

Synthesis of Di-tert-butyl (((S)-1-(tert-butoxy)-1,5-dioxo-5-((4-(tributylstannyl) phenethyl)amino)pentan-2-yl)carbamoyl)-L-glutamate (13). Referring to Scheme 13, a mixture of compound 12 (0.100 g, 0.20 mmol), TSTU (0.068 g, 0.22 mmol) and DIPEA (0.053 g, 0.41 mmol) were stirred in DMF (1 mL) at RT for 5 h. 2-(4-(tributylstannyl)phenyl)ethan-1-amine (Kurth et al., 1993) (0.083 g, 0.20 mmol) was added dropwise after dilution with DMF (1 mL). The reaction mixture was stirred overnight, concentrated and purified by silica gel flash chromatography eluting with 35% EtOAc/hexanes provided 0.067 g (37%) of oily material. ¹H NMR (500 MHz, CDCl₃) δ ppm 7.41 (d, J=10 Hz, 2H), 7.20 (d, J=5 Hz, 2H), 6.59 (m, 1H), 5.36-5.30 (m, 2H), 4.33 (m, 2H), 4.15 (m, 1H), 3.52-3.49 (m, 2H), 2.83-2.80 (m, 2H), 2.37-2.10 (m, 5H), 1.97-1.86 (m, 2H), 1.71-1.54 (m, 2H), 1.47 (m, 27H), 1.39-1.27 (m, 10H), 1.07-1.04 (m, 6H), 0.90 (m, 9H); ¹³C NMR (125 MHz, CDCl₃) δ ppm 172.5, 172.4, 171.9, 171.2, 157.3, 139.5, 138.7, 136.7, 128.8, 128.4, 82.2, 80.7, 60.4, 53.3, 53.1, 40.8, 35.7, 32.7, 31.6, 29.8, 29.1, 28.1, 28.0, 27.4, 27.2, 26.9, 21.1, 17.6, 14.2, 13.7, 13.6, 9.6. ESMS m/z: 880.4 (M+H)⁺, 904.3 (M+Na)⁺.

Radiosynthesis of (2S)-2-(3-(1-Carboxy-5-(4-[²¹¹At]asatatobenzamido)pentyl) ureido)pentanedioic acid ([²¹¹At]4), [²¹¹At]YC-I-27. Astatine was produced on a CS-30 cyclotron at Duke University and the NIH by bombarding natural bismuth metal targets with 28 MeV α-particles and isolated by dry distillation. The ²¹¹At was isolated in a solution of N-chlorosuccinimide (NCS) in MeOH (1 mg/mL).

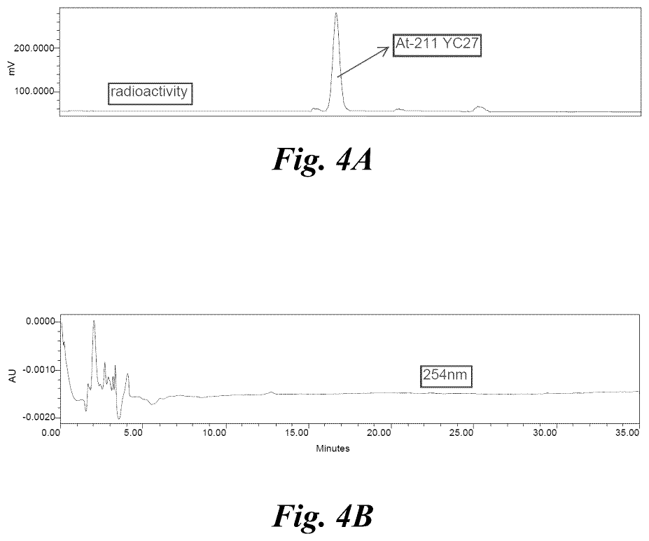

A solution of ²¹¹At in NCS/MeOH (74-370 MBq in 200-300 μL) and acetic acid (604) was added to 50 μg of stannane precursor. The reaction was allowed to proceed at 20° C. for 10 min, the MeOH was evaporated under a gentle stream of argon, and a solution of anisole in TFA (3% v/v; 100 μL) was added to the residue. The reaction mixture was allowed to stand for 30 min at 50° C. or 90 min at room temperature. The TFA was evaporated with argon and the compound reconstituted in 50 μL of 90:10 water: acetonitrile and injected onto a RP-HPLC column. The column was eluted at a flow rate of 1 mL/min with a gradient consisting of 0.1% TFA in H₂O (solvent A) and 0.1% TFA in acetonitrile (solvent B). Solvent B was kept at 5% for 5 min and then linearly increased to 100% over 30 min. Under those conditions, the product eluted with a t_{R} of ˜20 min. HPLC fractions containing the radiolabeled product were pooled, and most of the acetonitrile was evaporated under a stream of argon. The resultant solution was diluted with water (10 mL) and passed through either an activated C18 Sep-Pak plus cartridge or an Oasis HLB Sep-Pak cartridge (Waters). The cartridge was washed with 10 mL of water and the product eluted with 0.25 mL portions of ethanol. The fractions containing most of the radioactivity (typically 2-5) were pooled, the ethanol was evaporated, and the product was reconstituted in PBS. HPLC chromatogram of purified [²¹¹At]YC-I-27 after standing one hour in ethanol is shown FIG. 4A and FIG. 4B.

Radiosynthesis of (S)-2-(3-((R)-1-carboxy-2-(4-[¹²⁵I]iodobenzylthio)ethyl)ureido)-pentanedioic acid, [¹²⁵I]DCIBC. Referring now to Scheme 12, to a solution of (S)-bis(4-methoxybenzyl) 2-(3-((R)-1-((4-methoxybenzyl)oxy)-1-oxo-3-((4-(tributylstannyl)benzyl)thio)propan-2-yl)ureido)pentanedioate (0.1 mg) in 0.1 mL methanol was added 0.001 mL acetic acid, sodium [¹²⁵I] iodide followed by 0.005 mg N-chlorosuccinimide in 0.05 mL methanol solution. After 20 min at room temperature, the solvent was removed under a stream of N₂. A solution of 3% anisole in TFA (0.1 mL) was then added to the residue. After 5 min at room temperature, [¹²⁵I]DCIBC was isolated by HPLC (Econosil C18 10 μ, 250×4.6 mm, H₂O/CH₃CN/TFA (70/30/0.1), 1 ml/min, product peak eluting at 18 min). The radiochemical yield of the total synthesis was 53-59% (n=4). The specific activity was at least 1700 Ci/mmol.

### Example 3

### Results and Discussion

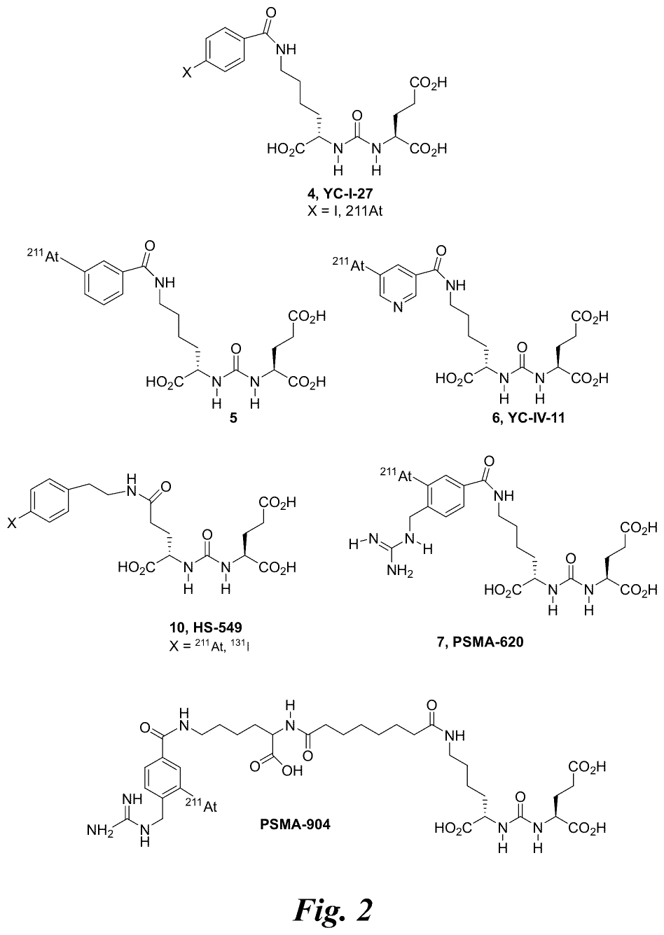

Synthesis of lysine-glutamate urea compounds. Lysine glutamate ureas compounds for radiotherapy are disclosed in FIG. 2. The lead compound in the lysine-glutamate ureas is YC-I-27. This radioiodinated compound has high and prolonged tumor uptake and a very high PSMA binding affinity Ki=0.01 nM (Chen et al., 2008). It has also been co-crystalized with PSMA, which indicates that the bulky iodo-phenyl moiety is accommodated by a hydrophobic auxiliary sub-pocket extending beyond the normal binding pocket and the additional hydrophobic-hydrophobic interactions accounts for the high binding affinity (Barinka et al., 2008).

Compounds 4 (YC-I-27 and 6 (YC-IV-11) were prepared as shown in Scheme 1 using stannane precursors (Chen 2008). Compounds 5, 7 (PSMA-602), and 8 can be prepared from precursor 9 and known stannanes also shown in Scheme 1 (Garg et al., 1991; Vaidyanathan and Zalutsky, 2007; Talanov et al., 2006.

Moreover, radiohalogenated prosthetic groups can also be conjugated to any of the previously reported linker-lysine glutamate urea linkers (compound 15 is used as an example) as outlined in Scheme 2 (Banerjee et al., 2008; Chen et al., 2012).

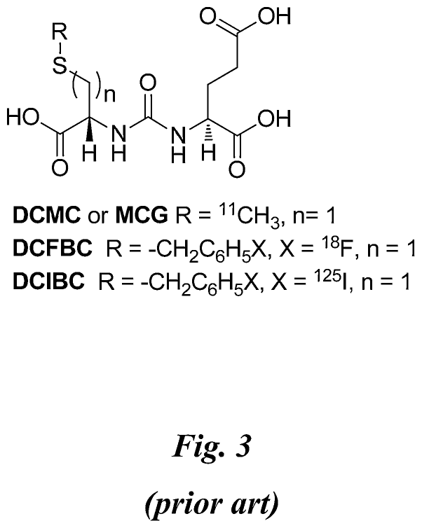

Synthesis of cysteine-glutamate urea compounds. Over the past 10 years, the cysteine-glutamate urea scaffold has been used for PSMA binding and imaging, starting with C-11 labeled DCMC (Pomper et al., 2002; Foss et al., 2005), continuing with F-18 labeled DCFBC (Mease et al., 2008; Cho et al., 2012) both for PET imaging with the latter currently in use in patients, and 1-125 labeled DCIBC (Dusich 2008) for SPECT imaging and or radiotherapy (FIG. 3).

Unlike the synthesis of [¹⁸F]DCFBC which required the synthesis of 4-[¹⁸F]benzylbromide for conjugation to the cysteine glutamate urea, the synthesis of [¹²⁵I]DCIBC utilizes a stannane derivative of the thio-benzoyl cysteine-glutamate urea, whose synthesis is outlined in Scheme 12. The stannane has been radioiodinated to produce [¹²⁵I]DCIBC (Dusich 2008) and identical chemistry can be utilized to prepare ²¹¹At labeled 14 as well as [¹³¹I]DCIBC.

Homologs of the cysteine-glutamate ureas where n=2 or 3 may have even higher binding affinity because the extended alkyl chain will permit a deeper penetration of the 4-halobenzyl group into the non-pharmacophore binding pocket. These compounds can be prepared analogously using Scheme 12 starting with commercial N^{α}-Fmoc-S-trityl-L-homocysteine and L-5-[5-trityl]-[N-9-fluorenylmethyloxycarbonyl]-mercaptonorvaline.

Synthesis of glutamate-glutamate urea compounds. Glutamate-glutamate ureas have been used to conjugate bulky radiometal chelating agents, fluorescent molecule, and chemotherapeutics. These compounds are too large to utilize the non-pharmacophore binding pocket and must utilize a void region to extend the bulky appendage outside of the protein. Novel compound 10 (HS-549, I or ²¹¹At labeled) which is similar in size to YC-I-27 can be prepared as outlined in Scheme 13. Compound 12 is prepared as reported (Kularatne et al., 2009), coupled to iodophenylethylamine and the esters hydrolyzed to give nonradioactive HS-549. Compound 12 is also coupled to 4-(tri-n-butylstannyl)phenethylamine (Kurth et al., 1993) to give compound 13 which is labeled with At-211 and the protecting groups removed as above to give [²¹¹At]10 ([²¹¹At]HS-549).

Radiosynthesis. The radiosynthesis of [²¹¹At]YC-I-27, [²¹¹At]YC-1V-11, [²¹¹At]HS-549, [²¹¹At]PSMA-620, and [²¹¹At]PSMA-904 were all performed using tri(n-butyl) stannane precursors following the procedure used for [²¹¹At]YC-I-27.

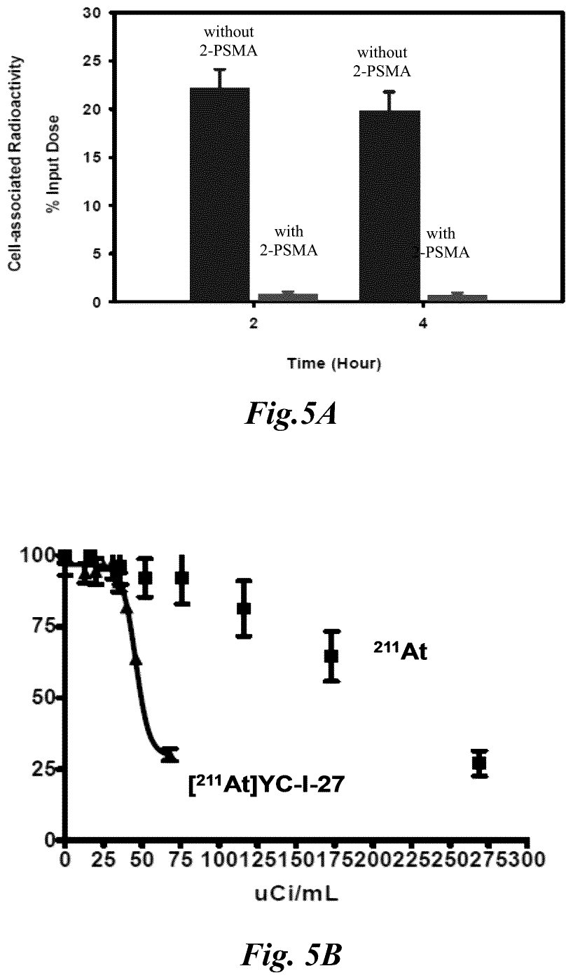

In vitro Radiotoxicity of [²¹¹At]4 ([²¹¹At]YC-I-27). FIG. 5A discloses the uptake of [²¹¹At]4 ([²¹¹At]YC-I-27) by LNCaP cells at 2 h and 4 h in the presence (right) and absence (left) of the known, high-affinity PSMA inhibitor 2-PSMA. Nearly all cell uptake of [²¹¹At]4 ([²¹¹At]YC-I-27) is blocked, indicating target (PSMA) specific binding. FIG. 5B discloses the cell kill due to [²¹¹At]4 ([²¹¹At]YC-I-27) vs. free [²¹¹At]astatide. These results show the capacity of [²¹¹At]4 ([²¹¹At]YC-I-27) to effectively kill cell relative to the untargeted alpha emitter ²¹¹At.

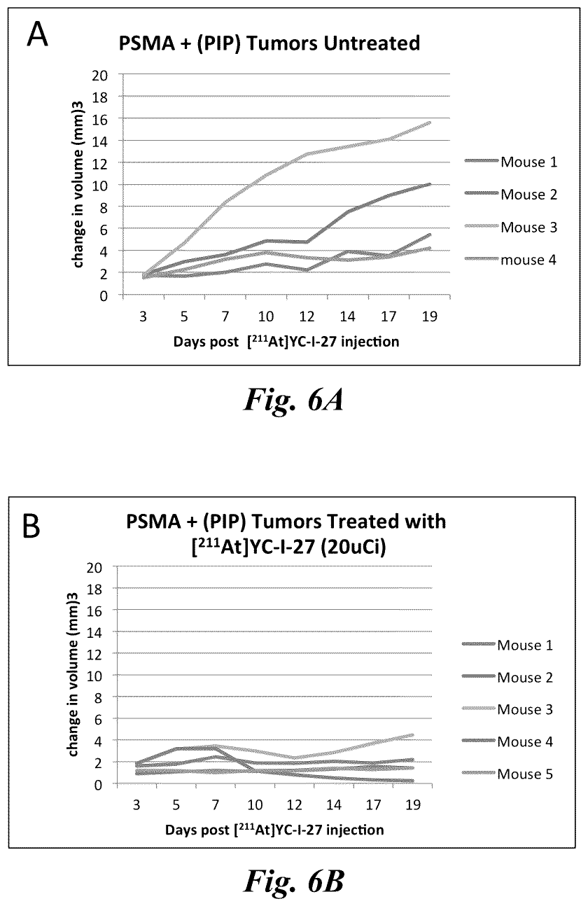

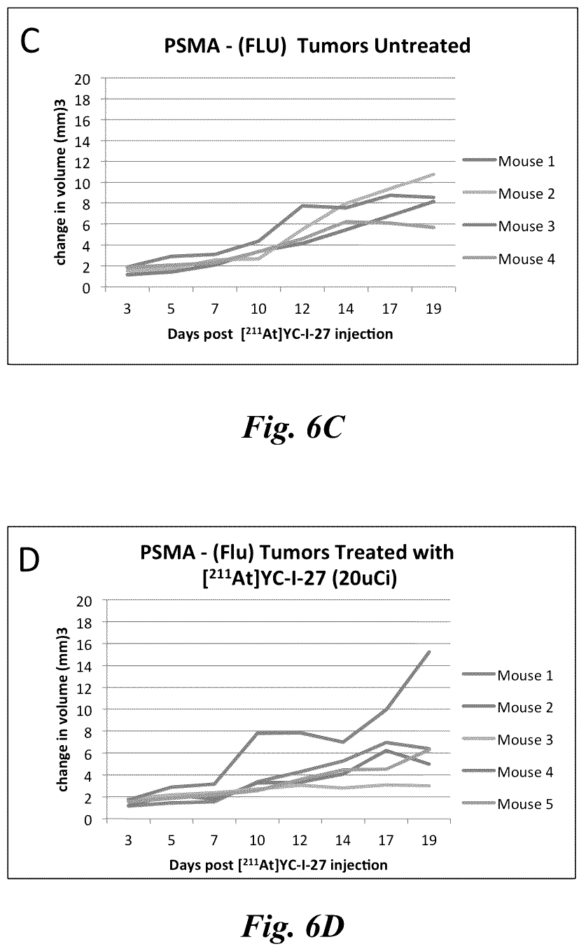

PSMA Specific Tumor Growth Inhibition from Treatment with [²¹¹At]4 ([²¹¹At]YC-I-27). A time-dependent growth inhibition study was performed for [²¹¹At]4 ([²¹¹At]YC-I-27) from 3 to 19 days after an single-bolus injection of [²¹¹At]4 ([²¹¹At]YC-I-27) on PSMA+(PIP) tumors and PSMA—(Flu) tumors (FIG. 6A and FIG. 6B). The same study was performed on PSMA+(PIP) tumors and PSMA—(Flu) tumors untreated (FIG. 6C and FIG. 6D).

Biodistribution of [¹³¹I] and [²¹¹At] labeled 4 (YC-I-27) in Mice. Table 2 and Table 3 show the organ % ID/g uptake values for [¹³¹I] and [²¹¹At] labeled 4 (YC-I-27) in selected organs in mice bearing PSMA+ and PSMA-tumor xenografts at 1, 2, 4 and 21 h, and 1, 2, 4 and 18 h post-injection, respectively.

TABLE 2
Biodistribution of [¹³¹I] labeled 4 (YC-I-27) in mice bearing
PSMA+ and PSMA− tumor xenografts (% ID/g)

| Organ | 1 H | 2 H | 4 H | 21 H |
|---|---|---|---|---|
| blood | 1.57 ± 0.69 | 0.86 ± 0.20 | 0.59 ± 0.27 | 0.16 ± 0.06 |
| heart | 1.88 ± 0.66 | 1.31 ± 0.58 | 0.87 ± 0.24 | 0.46 ± 0.22 |
| lung | 4.67 ± 0.63 | 4.92 ± 1.95 | 2.72 ± 1.01 | 1.63 ± 0.47 |
| liver | 7.37 ± 1.29 | 5.69 ± 1.49 | 3.21 ± 1.14 | 0.56 ± 0.09 |
| stomach | 0.92 ± 0.16 | 0.71 ± 0.46 | 0.69 ± 0.23 | 0.40 ± 0.07 |
| spleen | 21.2 ± 3.62 | 26.25 ± 15.11 | 18.2 ± 6.19 | 5.25 ± 1.51 |
| thyroid | 0.45 ± 0.14 | 0.24 ± 0.18 | 0.10 ± 0.22 | 0.13 ± 0.11 |
| kidney | 118 ± 16.9 | 119 ± 24.9 | 117 ± 32.7 | 126 ± 28.4 |
| muscle | 0.73 ± 0.22 | 0.62 ± 0.19 | 0.43 ± 0.30 | 0.14 ± 0.03 |
| Sm int. | 1.83 ± 0.41 | 1.92 ± 0.55 | 1.37 ± 0.51 | 0.33 ± 0.12 |
| bladder | 2.99 ± 0.42 | 2.96 ± 1.12 | 1.37 ± 0.48 | 0.48 ± 0.09 |
| PC-3 PiP | 13.1 ± 5.55 | 15.5 ± 4.19 | 16.3 ± 3.80 | 25.6 ± 10.2 |
| PC-3 flu | 0.62 ± 0.11 | 0.40 ± 0.11 | 0.22 ± 0.07 | 0.06 ± 0.01 |

TABLE 3
Biodistribution of [²¹¹At] labeled 4 (YC-I-27) in mice bearing
PSMA+ and PSMA− tumor xenografts (% ID/g)

| Organ | 1 H | 2 H | 4 H | 18 H |
|---|---|---|---|---|
| blood | 1.67 ± 0.32 | 1.26 ± 0.15 | 0.99 ± 0.12 | 0.53 ± 0.05 |
| heart | 2.36 ± 0.48 | 1.79 ± 0.31 | 1.65 ± 0.39 | 1.10 ± 0.16 |
| lung | 5.82 ± 1.28 | 6.30 ± 1.57 | 5.54 ± 1.79 | 3.61 ± 0.40 |
| liver | 2.11 ± 0.65 | 1.58 ± 0.21 | 1.63 ± 0.10 | 0.82 ± 0.07 |
| stomach | 10.1 ± 1.66 | 9.29 ± 2.86 | 13.3 ± 3.12 | 9.42 ± 3.04 |
| spleen | 29.2 ± 10.2 | 20.3 ± 5.21 | 20.3 ± 3.55 | 8.01 ± 2.04 |
| thyroid | 3.68 ± 1.10 | 3.56 ± 0.95 | 3.83 ± 1.18 | 6.50 ± 1.97 |
| kidney | 71.5 ± 12.0 | 60.2 ± 6.16 | 60.2 ± 11.5 | 57.4 ± 7.36 |
| muscle | 0.95 ± 0.15 | 0.81 ± 0.12 | 0.72 ± 0.14 | 0.49 ± 0.27 |
| Sm int. | 3.67 ± 0.65 | 2.08 ± 0.41 | 1.85 ± 0.23 | 1.08 ± 0.10 |
| bladder | 5.45 ± 2.57 | 4.55 ± 0.86 | 4.17 ± 0.74 | 2.51 ± 0.39 |
| PC-3 PiP | 17.9 ± 2.98 | 20.7 ± 3.42 | 18.3 ± 2.90 | 31.1 ± 9.78 |
| PC-3 flu | 2.18 ± 0.44 | 1.81 ± 0.27 | 1.53 ± 0.22 | 1.17 ± 0.20 |

Biodistribution of S)-2-(3-((R)-1-carboxy-2-(4-[¹²⁵I]iodobenzylthio)ethyl) ureido)-pentanedioic acid, [¹²⁵I]DCIBC. Table 4 shows the biodistribution of [¹²⁵I] labeled DCIBC in selected organs in mice bearing PSMA+ and PSMA-tumor xenografts (% ID/g) at 1, 2, and 5 h post-injection.

TABLE 4
Biodistribution of [¹²⁵I] labeled DCIBC in mice bearing
PSMA+ and PSMA− tumor xenografts (% ID/g)

| Organ | 1 H | 2 H | 5 H |
|---|---|---|---|
| Blood | 5.8 ± 2.3 | 4.9 ± 2.6 | 3.3 ± 1.1 |
| Heart | 1.5 ± 0.2 | 1.1 ± 0.2 | 0.9 ± 0.5 |
| Lung | 2.2 ± 0.4 | 1.9 ± 0.5 | 1.0 ± 0.5 |
| Liver | 20 ± 4 | 17 ± 4 | 17 ± 1 |
| Spleen | 5.1 ± 2.2 | 3.2 ± 0.6 | 3.9 ± 1.1 |
| Kidney | 77 ± 17 | 30 ± 8 | 18 ± 6 |
| Sm Int. | 1.2 ± 0.3 | 1.1 ± 0.2 | 1.7 ± 1.5 |
| Lrg Int. | 1.2 ± 0.1 | 1.2 ± 0.5 | 2.7 ± 1.9 |
| Muscle | 1.0 ± 0.8 | 0.5 ± 0.2 | 0.4 ± 0.2 |
| PSMA+ tumor | 8.0 ± 1.3 | 5.6 ± 1.3 | 5.5 ± 0.6 |
| PSMA− tumor | 1.3 ± 0.2 | 0.9 ± 0.1 | 1.0 ± 0.3 |

Biodistribution of [²¹¹At] and [¹³¹I] labeled PSMA-904 in Mice. Table 5 and Table 6 show the biodistribution of [²¹¹At] and [¹³¹I] labeled PSMA-904 respectively, in selected organs in mice bearing PSMA+ and PSMA-tumor xenografts (% ID/g) at 1, 2, and 21 h post-injection.

TABLE 5
Biodistribution of [¹³¹I] labeled PSMA-904 in mice bearing
PSMA+ PC-3 PiP and PSMA− PC-3 flu tumor xenografts (% ID/g)

| Organ | 1 H | 2 H | 21 H |
|---|---|---|---|
| blood | 0.46 ± 0.17 | 0.18 ± 0.07 | 0.02 ± 0.01 |
| heart | 0.46 ± 0.18 | 0.27 ± 0.14 | 0.02 ± 0.01 |
| lung | 1.39 ± 0.36 | 0.81 ± 0.37 | 0.03 ± 0.01 |
| liver | 5.16 ± 3.81 | 3.48 ± 2.35 | 0.24 ± 0.39 |
| stomach | 0.73 ± 0.26 | 1.03 ± 0.62 | 1.98 ± 3.10 |
| spleen | 9.83 ± 3.28 | 3.41 ± 2.01 | 0.16 ± 0.07 |
| thyroid | 0.11 ± 0.13 | 0.31 ± 0.23 | 0.42 ± 0.35 |
| kidney | 121 ± 23.8 | 110 ± 37.2 | 3.03 ± 2.96 |
| muscle | 0.31 ± 0.14 | 0.13 ± 0.03 | 0.03 ± 0.02 |
| Sm int. | 33.81 ± 6.36 | 14.2 ± 12.0 | 0.87 ± 0.92 |
| Lrg Int. | 0.50 ± 0.65 | 21.3 ± 15.9 | 14.5 ± 8.08 |
| bladder | 0.71 ± 0.23 | 0.64 ± 0.25 | 0.07 ± 0.09 |
| PC-3 PiP | 27.6 ± 7.59 | 24.7 ± 9.77 | 19.2 ± 7.28 |
| PC-3 flu | 0.49 ± 0.23 | 0.22 ± 0.06 | 0.02 ± 0.01 |

TABLE 6
Biodistribution of [²¹¹At] labeled PSMA-904 in mice bearing
PSMA+ PC-3 PiP and PSMA− PC-3 flu tumor xenografts (% ID/g)

| Organ | 1 H | 2 H | 21 H |
|---|---|---|---|
| blood | 0.65 ± 0.33 | 0.35 ± 0.03 | 0.48 ± 0.13 |
| heart | 0.84 ± 0.27 | 0.76 ± 0.11 | 1.12 ± 0.27 |
| lung | 2.24 ± 0.51 | 1.96 ± 0.26 | 2.89 ± 0.93 |
| liver | 5.11 ± 4.13 | 3.80 ± 2.39 | 0.59 ± 0.21 |
| stomach | 1.64 ± 0.62 | 2.86 ± 0.67 | 9.36 ± 2.18 |
| spleen | 9.51 ± 2.76 | 4.31 ± 1.96 | 2.07 ± 0.68 |
| thyroid | 0.51 ± 0.16 | 1.03 ± 0.26 | 3.74 ± 1.42 |
| kidney | 86.5 ± 15.5 | 87.7 ± 11.0 | 4.38 ± 3.46 |
| muscle | 0.38 ± 0.16 | 0.21 ± 0.03 | 0.22 ± 0.06 |
| Sm int. | 33.9 ± 5.49 | 13.8 ± 10.9 | 1.51 ± 0.91 |
| Lrg Int. | 0.75 ± 0.98 | 26.7 ± 15.3 | 8.55 ± 5.07 |
| bladder | 1.17 ± 0.20 | 1.30 ± 0.24 | 1.99 ± 0.54 |
| PC-3 PiP | 22.7 ± 5.42 | 21.1 ± 6.16 | 12.1 ± 5.03 |
| PC-3 flu | 0.80 ± 0.30 | 0.52 ± 0.05 | 0.84 ± 0.23 |

Biodistribution of [²¹¹At] Labeled 6 (YC-IV-11) in Mice. Table 7 shows the biodistribution of [²¹¹At] labeled 6 (YC-IV-11) in selected organs in mice bearing PSMA+ and PSMA-tumor xenografts (% ID/g) at 1, 2, 4, and 21 h post-injection.

TABLE 7
Biodistribution of [¹³¹I] labeled 6 (YC-IV-11) in mice bearing PSMA+
PC-3 PiP and PSMA− PC-3 flu tumor xenografts (% ID/g)

| Organ | 1 H | 2 H | 4 H | 21 H |
|---|---|---|---|---|
| blood | 1.80 ± 1.57 | 0.64 ± 0.11 | 0.61 ± 0.12 | 0.30 ± 0.07 |
| heart | 1.67 ± 0.27 | 1.25 ± 0.28 | 1.27 ± 0.30 | 0.91 ± 0.33 |
| lung | 5.48 ± 1.26 | 4.62 ± 1.06 | 3.72 ± 0.98 | 2.89 ± 1.12 |
| liver | 1.56 ± 0.35 | 1.20 ± 0.27 | 0.79 ± 0.17 | 0.46 ± 0.12 |
| stomach | 5.37 ± 0.61 | 6.24 ± 2.99 | 8.03 ± 2.26 | 4.30 ± 1.24 |
| spleen | 8.87 ± 1.33 | 8.00 ± 2.08 | 5.83 ± 1.91 | 4.23 ± 0.84 |
| thyroid | 1.64 ± 0.25 | 1.85 ± 0.36 | 2.02 ± 0.27 | 2.22 ± 1.04 |
| kidney | 135 ± 18.9 | 124 ± 23.6 | 69.6 ± 43.4 | 5.37 ± 15.5 |
| muscle | 0.53 ± 0.15 | 0.43 ± 0.11 | 0.40 ± 0.21 | 0.26 ± 0.10 |
| Sm int. | 1.48 ± 0.29 | 1.21 ± 0.42 | 0.95 ± 0.21 | 0.63 ± 0.13 |
| bladder | 2.88 ± 0.37 | 3.08 ± 0.92 | 2.84 ± 0.76 | 1.72 ± 0.65 |
| PC-3 PiP | 13.8 ± 5.06 | 15.2 ± 4.68 | 13.3 ± 4.19 | 12.34 ± 3.01 |
| PC-3 flu | 1.38 ± 0.22 | 1.19 ± 0.24 | 1.12 ± 0.20 | 0.57 ± 0.19 |

Biodistribution of [²¹¹At] labeled 7 (PSMA-620) in Mice. Table 8 shows the biodistribution of [²¹¹At] labeled 7 (PSMA-620) in selected organs in mice bearing PSMA+ and PSMA-tumor xenografts (% ID/g) at 1, 2, 4, 14, and 21 h post-injection.

TABLE 8
Biodistribution of [¹³¹I] labeled 7 (PSMA-620) in mice bearing
PSMA+ PC-3 PiP and PSMA− PC-3 flu tumor xenografts (% ID/g)

| Organ | 1 H | 2 H | 4 H | 14 H | 21 H |
|---|---|---|---|---|---|
| blood | 0.95 ± 0.15 | 0.58 ± 0.08 | 0.30 ± 0.08 | 0.16 ± 0.04 | 0.09 ± 0.07 |
| heart | 1.29 ± 0.37 | 0.89 ± 0.18 | 0.75 ± 0.34 | 0.48 ± 0.12 | 0.40 ± 0.11 |
| lung | 3.29 ± 0.56 | 1.99 ± 0.23 | 1.69 ± 0.67 | 1.02 ± 0.27 | 1.00 ± 0.21 |
| liver | 8.25 ± 2.47 | 5.62 ± 1.17 | 4.37 ± 2.96 | 0.60 ± 0.28 | 0.33 ± 0.06 |
| stomach | 2.01 ± 0.41 | 2.55 ± 0.69 | 2.87 ± 0.55 | 2.24 ± 1.08 | 1.91 ± 0.95 |
| spleen | 17.1 ± 3.71 | 6.81 ± 3.20 | 3.63 ± 2.40 | 1.27 ± 0.42 | 0.93 ± 0.37 |
| thyroid | 0.79 ± 0.17 | 0.77 ± 0.25 | 0.75 ± 0.17 | 0.74 ± 0.27 | 0.88 ± 0.13 |
| kidney | 103 ± 24.0 | 89.0 ± 5.18 | 77.7 ± 38.8 | 17.2 ± 10.7 | 7.52 ± 1.82 |
| muscle | 0.56 ± 0.05 | 0.34 ± 0.08 | 0.23 ± 0.10 | 0.10 ± 0.02 | 0.09 ± 0.01 |
| Sm int. | 3.14 ± 0.66 | 2.29 ± 0.69 | 1.32 ± 0.68 | 0.42 ± 0.12 | 0.31 ± 0.07 |
| bladder | 1.99 ± 0.50 | 1.65 ± 0.48 | 1.96 ± 1.10 | 0.74 ± 0.35 | 0.56 ± 0.37 |
| PC-3 PiP | 16.5 ± 4.78 | 17.2 ± 4.34 | 18.3 ± 4.27 | 15.8 ± 2.42 | 13.6 ± 3.33 |
| PC-3 flu | 1.09 ± 0.21 | 0.80 ± 0.11 | 0.57 ± 0.21 | 0.28 ± 0.10 | 0.20 ± 0.08 |

Biodistribution of [¹³¹I] and [²¹¹At] labeled 10 (HS-549) in Mice. Table 9 and Table show the biodistribution of [¹³¹I] and [²¹¹At] labeled 10 (HS-549) in selected organs in mice bearing PSMA+ and PSMA-tumor xenografts (% ID/g) at 1, 2, and 21 h post-injection.

TABLE 9
Biodistribution of [¹³¹I] labeled 10 (HS-549) in mice bearing PSMA+
PC-3 PiP and PSMA− PC-3 flu tumor xenografts (% ID/g)

| Organ | 1 H | 2 H | 21 H |
|---|---|---|---|
| blood | 5.90 ± 1.05 | 2.92 ± 0.32 | 1.39 ± 0.35 |
| heart | 2.30 ± 0.63 | 1.14 ± 0.18 | 0.41 ± 0.09 |
| lung | 4.60 ± 1.09 | 2.73 ± 0.66 | 0.87 ± 0.27 |
| liver | 21.2 ± 4.14 | 26.0 ± 3.09 | 1.86 ± 0.51 |
| stomach | 0.92 ± 0.20 | 0.47 ± 0.17 | 0.14 ± 0.04 |
| spleen | 6.76 ± 3.42 | 2.71 ± 0.85 | 0.29 ± 0.12 |
| thyroid | 1.17 ± 0.43 | 0.69 ± 0.29 | 0.36 ± 0.05 |
| kidney | 106 ± 18.9 | 96.6 ± 39.1 | 3.13 ± 1.65 |
| muscle | 0.86 ± 0.26 | 0.46 ± 0.12 | 0.16 ± 0.04 |
| Sm int. | 1.98 ± 0.30 | 2.42 ± 0.40 | 0.37 ± 0.06 |
| Lrg Int. | 0.52 ± 0.12 | 1.32 ± 0.25 | 0.63 ± 0.39 |
| bladder | 4.34 ± 2.16 | 2.34 ± 1.04 | 0.65 ± 0.20 |
| PC-3 PiP | 78.1 ± 19.1 | 74.7 ± 13.1 | 22.6 ± 22.1 |
| PC-3 flu | 2.21 ± 0.53 | 1.51 ± 0.30 | 0.44 ± 0.18 |

TABLE 10
Biodistribution of [²¹¹At] labeled 10 (HS-549) in mice bearing
PSMA+ PC-3 PiP and PSMA− PC-3 flu tumor xenografts (% ID/g)

| Organ | 1H | 2H | 21H |
|---|---|---|---|
| blood | 5.53 ± 0.84 | 2.87 ± 0.40 | 0.92 ± 0.15 |
| heart | 4.34 ± 0.96 | 3.36 ± 0.59 | 1.64 ± 0.54 |
| lung | 9.77 ± 1.75 | 8.24 ± 2.25 | 4.26 ± 0.72 |
| liver | 7.94 ± 2.15 | 4.65 ± 1.28 | 0.89 ± 0.13 |
| stomach | 7.07 ± 2.24 | 14.3 ± 3.18 | 12.6 ± 6.20 |
| spleen | 8.79 ± 2.09 | 6.13 ± 1.59 | 2.31 ± 0.67 |
| thyroid | 3.24 ± 0.56 | 4.11 ± 2.0 | 3.78 ± 0.63 |
| kidney | 46.7 ± 8.24 | 40.4 ± 15.5 | 2.60 ± 0.77 |
| muscle | 1.11 ± 0.29 | 0.76 ± 0.18 | 0.29 ± 0.09 |
| Sm int. | 5.52 ± 0.82 | 3.81 ± 1.24 | 1.28 ± 0.30 |
| Lrg Int. | 1.16 ± 0.20 | 4.59 ± 0.81 | 0.93 ± 0.41 |
| bladder | 5.85 ± 1.62 | 5.04 ± 0.87 | 2.78 ± 0.82 |
| PC-3 PiP | 43.25 ± 9.81 | 42.0 ± 7.16 | 10.6 ± 9.91 |
| PC-3 flu | 3.48 ± 0.53 | 2.82 ± 0.60 | 1.15 ± 0.32 |

Comparative analysis of [¹³¹I/²¹¹At] YC-I-27 and [¹³¹I/²¹¹At]HS-549. As outlined on Scheme 1 and Scheme 4 respectively, compound YC-I-27 and compound HS-549 were both prepared from stannane precursors. They have the same molecular weight and measured Ki (Table 11). These two compounds only differ in structure in the non-pharmacophore binding pocket, and their structures overlap exactly in docking studies (PSMA binding site).

TABLE 11
Physical properties of compounds [I]-YC-I-27 and [I]-HS-549

| Name | YC-I-27 | HS-549 |
|---|---|---|
| Structure | 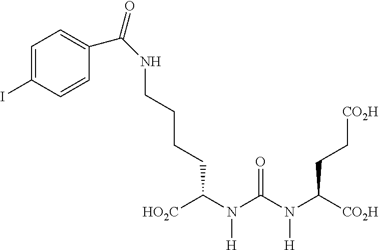 [SMILES: O=C(O)CC[C@H](NC(=O)N[C@@H](CCCCNC(=O)c1ccc(I)cc1)C(=O)O)C(=O)O] | 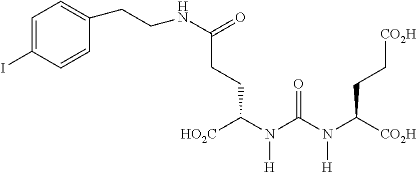 [SMILES: O=C(O)CC[C@H](NC(=O)N[C@@H](CCC(=O)NCCc1ccc(I)cc1)C(=O)O)C(=O)O] |
| Chemical | C₁₉H₂₄IN₃O₈ | C₁₉H₂₄IN₃O₈ |
| Formula | | |
| Molecular | 549.31 | 549.31 |
| Weight | | |
| (g/mol) | | |
| Ki (nM) | 0.01 | 0.01 |

The biodistributions of [¹³¹I] YC-I-27, [²¹¹At] YC-I-27, [¹³¹I] HS-549 and [²¹¹At]HS-549 have been assessed and they all demonstrated PSMA positive tumor uptake as outlined in Tables 2, 3, 9, and 10, respectively.

Tumor to kidney ratios are given for compound [¹³¹I]W-1-27, [²¹¹At]YC-I-27, [¹³¹I]HS-549, and [²¹¹At]HS-549 at 2H and 21H post-injection (Table 12).

TABLE 12
Comparative data for tumor/kidney ratios for
[¹³¹I/²¹¹At] YC-I-27 and [¹³¹I/²¹¹At]HS-549

| Compound | 2 H | 21 H |
|---|---|---|
| [¹³¹I]YC-I-27 | 0.1 | 0.2 |
| [¹³¹I]HS-549 | 0.8 | 7.2 |
| [²¹¹At]YC-I-27 | 0.3 | 0.5 |
| [²¹¹At]HS-549 | 1.0 | 4.1 |

Unlike [¹³¹I]Y C-I-27 and [²¹¹At]YC-I-27, compounds [¹³¹I]HS-549 and [²¹¹At]HS-549 clear from the kidneys giving higher tumor to kidney ratios.

Further, [²¹¹At]HS-549 has higher uptake in non-target organs stomach, spleen and thyroid compared to [¹³¹I]HS-549, indicating some instability of the At-211 label, however, these uptakes are lower than those seen with [²¹¹At]C-I-27 (Table 13).

TABLE 13
Comparative biodistribution data for non-target organs for
[¹³¹I]HS-549, and [²¹¹At]HS-549 at 21 H post-injection and
[²¹¹At]YC-I-27 at 18 H post-injection (% D/g)

| Non-target organs | [¹³¹I]HS-549 | [²¹¹At]HS-549 | [²¹¹At]YC-I-27 |
|---|---|---|---|
| stomach | 0.14 | 12.6 | 9.4 |
| spleen | 0.3 | 2.2 | 8.0 |
| thyroid | 0.36 | 3.8 | 6.5 |

In summary, [²¹¹At]HS-549 exhibited faster renal clearance and lower normal tissue uptake than YC-I-27, making [²¹¹At]HS-549 a promising At-211 labeled agent for PSMA targeted radiotherapy.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
International PCT Patent Application Publication No. PCT/US03/00680 to Pomper M G, Zhang J, Kozikowski A P, Musachio J L. for Imaging agents and methods of imaging NAALADase of PSMA, published 2003 Jul. 24 (WO 03/60523 A1);
International PCT Patent Application Publication No. PCT/US2008/007947 to Pomper, M. G., Ray, S., Mease, R. C., Foss, C. for Labeled inhibitors of prostate specific membrane antigen (PSMA), biological evaluation, and use as imaging agents, published 2008 Dec. 31 (WO 2009/002529 A2);
International PCT Patent Application Publication No. PCT/US2009/052456 to Pomper M G, Mease R C, Chen Y. for Preparation of glutamic acid heterodimer prostate-specific membrane antigen PSMA binding agents for therapeutic and imaging use, published 2010 Feb. 4 (WO 2010/014933 A2);
International PCT Patent Application Publication No. PCT/US2010/028020 to Pomper, Martin G.; Mease, Ronnie C.; Ray, Sangeeta; Chen, Ying. for PSMA-targeting compounds and uses thereof, published 2010 Sep. 23 (WO 2010/108125 A2);
International PCT Patent Application Publication No. PCT/US2012/067162 to Pomper, Martin G. Mease, Ronnie C., Ray, Sangeeta, Shallal, Hassan for Homomultivalent and heteromultivalent inhibitors of prostate specific membrane antigen (PSMA) and uses thereof, published 2013 Jun. 6 (WO2013/082338 A1);
International PCT Patent Application Publication No. PCT/US2011/026238 to Low P. S., Chelvam V., Kim Y. for PSMA binding ligand-linker conjugates and methods for using, published 2011 Sep. 1 (WO2011/106639);
International PCT Patent Application Publication No. PCT/US2009/061067 to Low P. S., Chelvam V., Kim Y. for PSMA binding ligand-linker conjugates and methods for using, published 2010 Apr. 22 (WO 2010/045598 A2);
International PCT Patent Application Publication No. PCT/US2008/073375 to Low P. S., Chelvam V., Kim Y. for PSMA binding ligand-linker conjugates and methods for using, published 209/02/26 (WO 2009/026177 A1);
U.S. Patent Application Publication No. US 2008/0193381 A1 to Babich J W, Zimmerman C N, Maresca K P. for Heterodimers of glutamic acid, published 2008 Aug. 14 (Ser. No. 11/936,659);
U.S. Patent Application Publication No. US 2013/0034494 A1 to Babich J W, Zimmerman C, Joyal J L, Lu G. for Radiolabeled prostate specific membrane antigen inhibitors, published 2013 Feb. 7 (Ser. No. 13/566,674);
Banerjee S R, Foss C A, Castanares M, Mease R C, Byun Y, Fox J J, Hilton J, Lupold S, Kozikowski A P, Pomper M G. Synthesis and evaluation of technetium-99m- and rhenium-labeled inhibitors of the prostate-specific membrane antigen (PSMA). J Med Chem 2008; 51:4504-4517;
Barinka C, Byun Y, Dusich C L, Ray S, Chen Y, Castanares M, Kozikowski A P, Mease R C, Pomper M G, Lubkowski J. Interactions between Human Glutamate Carboxypeptidase II and Urea-based Inhibitors: Structural Characterization. J Med Chem, 2008; 51:7737-7743;
Chang, S. S. et al. Five different anti-prostate-specific membrane antigen (PSMA) antibodies confirm PSMA expression in tumor-associated neovasculature. Cancer Res. 1999, 59, 3192;
Chen Y, Foss C A, Byun Y, Nimmagadda S, Pullambhatla M, Fox J J, Castanares S E, Babich J W, Mease R C, Pomper M G. Radiohalogenated PSMA-based ureas as imaging agents for prostate cancer. J Med Chem 2008; 51:7933-7943;
Chen Y, Pullambhatla M, Banerjee S R, Byun Y, Stathis M, Rojas C, Slusher B S, Mease R C, Pomper M G. 2012; 23(12) 2377-2385;
Cho S Y, Gage K L, Mease R C, et al. Biodistribution, tumor detection, and radiation dosimetry of 18F-DCFBC, a low molecular weight inhibitor of PSMA, in patients with metastatic prostate cancer. J. Nucl. Med. 2012; 53:1883-1891.
Dusich C L. Imaging prostate cancer: Design, synthesis and biological evaluation of optical and radioactive prostate-specific membrane antigen (PSMA) inhibitors. PhD Thesis Johns Hopkins University 2008.
Foss C A, Mease R C, Fan H, Wang Y, Ravert H T, Dannals R F, Olszewski R T, Heston W D, Kozikowski A P, Pomper M G. Radiolabeled small molecule ligands for prostate-specific membrane antigen: in vivo imaging in experimental models of prostate cancer. Clin Cancer Res 2005; 11:4022-4028.
Garg S, Garg P K, Zalutsky M R. N-succinimidyl 5-(trialkylstannyl)-3-pyridinecarboxylates: a new class of reagents for protein radioiodination. Bioconjugate Chem. 1991; 2:50-56;
Huang, X.; Bennett, M.; Thorpe, P. E. Prostate 2004, 61, 1;
Kiess A P, Minn I, Vaidyanathan G, Hobbs R F, Josefsson A, Shen C, Brummet M, Chen Y, Choi J, Koumarianou E, Baidoo K, Brechbiel M W, Mease R C, Sgoouros G, Zalutsky M R, Pomper M G. (2S0-2-(3-(1-Carboxy-5-(4-[211At]astatobenzamido)pentyl)ureido)-pentanedioic acid for PSMA-targeted α-particle radiopharmaceutical therapy. J. Nuc. Med. doi:10.2967/jnumed.116.174300;
Kularatne S A, Zhou Z, Yang J, Post C B, Low P S. Design, synthesis, and preclinical evaluation of prostate-specific membrane antigen (PSMA)-targeted-99mTc radioimaging agents. Molecular Pharmaceutics 2009; 6(3) 790-800;
Kurth M, Pelegrin A, Rose, K, Offord R E, Pochon S, Mach J-P, Buchegger F. Site-specific conjugation of a radioiodinated phenethylamine derivative to a monoclonal antibody results in increased radioactivity localization in tumor. J. Med. Chem. 1993; 36:1255-1261;
Liu, H.; Moy, P.; Kim, S.; Xia, Y.; Rajasekaran, A.; Navarro, V.; Knudsen, B.; Bander, N. H. Cancer Res. 1997, 57, 3629;
Mease R C, Dusich C L, Foss C A, Ravert H T, Dannals R F, Seidel J, Prideaux A, Fox J J, Sgouros G, Kozikowski A P, Pomper M G. Synthesis and in vivo evaluation of N-[N-[(S)-1,3-dicarboxypropyl]carbamoyl]-4-[18F]fluorobenzyl-L-cysteine, [18F]DCFBC: a new imaging probe for prostate cancer. Clin Cancer Res 2008; 14:3036-3043;
Pomper M G, Musachio J L, Zhang J, Zhou Y, Scheffel U, Hilton J, Maini A, Dannals R F, Wong D F, Kozikowski A P. 11C-MCG: Synthesis, uptake selectivity and primate PET of a probe for glutamate carboxypeptidase II (NAALADase.) Mol Imaging 2002; 1:96-101;
Schuelke, N.; Varlamova, O. A.; Donovan, G. P.; Ma, D.; Gardner, J. P.;
Morrissey, D. M.; Arrigale, R. R.; Zhan, C.; Chodera, A. J.; Surowitz, K. G.; Maddon, P. J.; Heston, W. D. W.; Olson, W. C. Proc. Natl. Acad. Sci. 2003, 100, 12590;
Talanov, V S, Garmestani K, Regino C A S, Milenic D E, Plascjak P S, Waldmann T A, Brechbiel M W. Preparation and in vivo evaluation of a novel stabilized linker for 211At labeling of protein. Nucl. Med. Biol. 2006; 33:469-480;
Vaidyanathan G, Zalutsky M R. Synthesis of N-succinimidyl 4-guanidinomethyl-3-[*I]iodobenzoate: a radio-iodination agent for labeling internalizing proteins and peptides. Nat Protoc 2007; 2:282-286.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 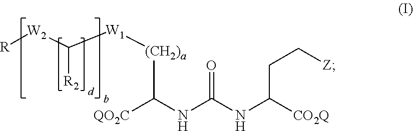
wherein:
Z is tetrazole or CO₂Q;
Q is H or a protecting group;
a is an integer selected from the group consisting of 0, 1, 2, 3, 4, 5, 6, 7, and 8;
W₁ is selected from the group consisting of —C(═O)—NR₁—, —NR₁—C(═O)—, and —S—;
each R₁ is independently H or a C₁-C₆ alkyl;
each R₂ is independently H or —COOR₃;
each R₃ is independently H, C₁-C₆ alkyl, C₆-C₁₂ aryl or C₄-C₁₆ alkylaryl;
b is an integer selected from the group consisting of 0, 1, 2, and 3;
d is an integer selected from the group consisting of 1, 2, 3, 4, 5, 6, 7, and 8;
each W₂ is independently selected from the group consisting of —C(═O)—NR₁— and —NR₁—C(═O)—;
R is selected from the group consisting of: 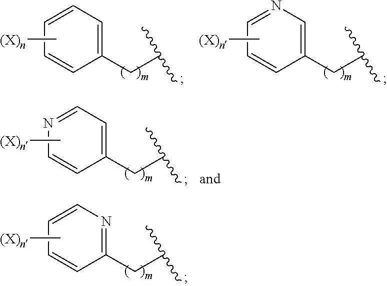
wherein X is selected from the group consisting of iodine, astatine, a bromine, a radioisotope of iodine, a radioisotope of astatine, a radioisotope of bromine, Sn(R₄)₃, Si(R₄)₃, Hg(R₄), B(OH)₂, —NHNH₂, —CH₂—NH—C(═NH)—NH₂;
R₄ is C₁-C₆ alkyl;
m is an integer selected from the group consisting of 0, 1, 2, 3, 4, 5, 6, 7, and 8;
n is an integer selected from the group consisting of 1, 2, 3, 4, and 5;
n′ is an integer selected from the group consisting of 1, 2, 3, and 4;
and stereoisomers and pharmaceutically acceptable salts thereof.

2. The compound of claim 1, wherein the compound of formula (I) is selected from the group consisting of: 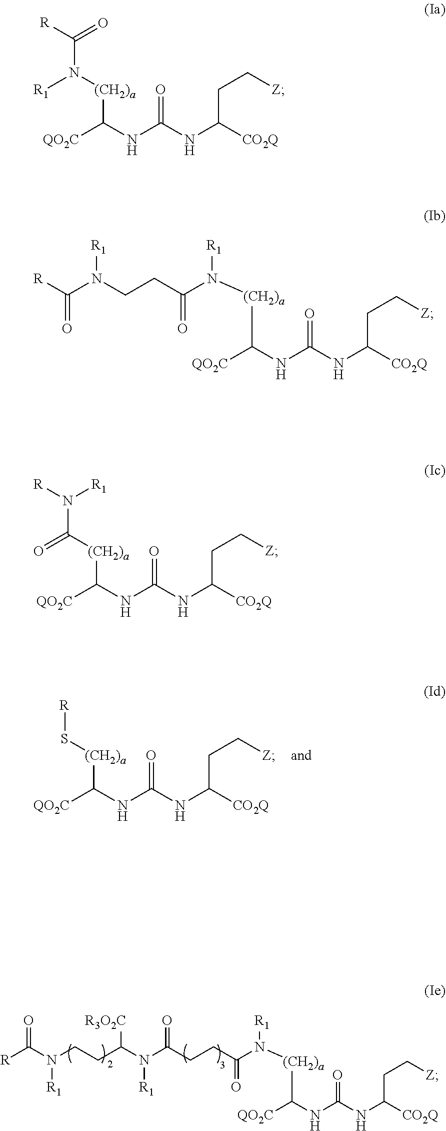
wherein Z, Q, R, R₁, R₃, a are defined as above;
and stereoisomers and pharmaceutically acceptable salts thereof.

3. The compound of claim 1, wherein the compound of formula (I) is selected from the group consisting of: 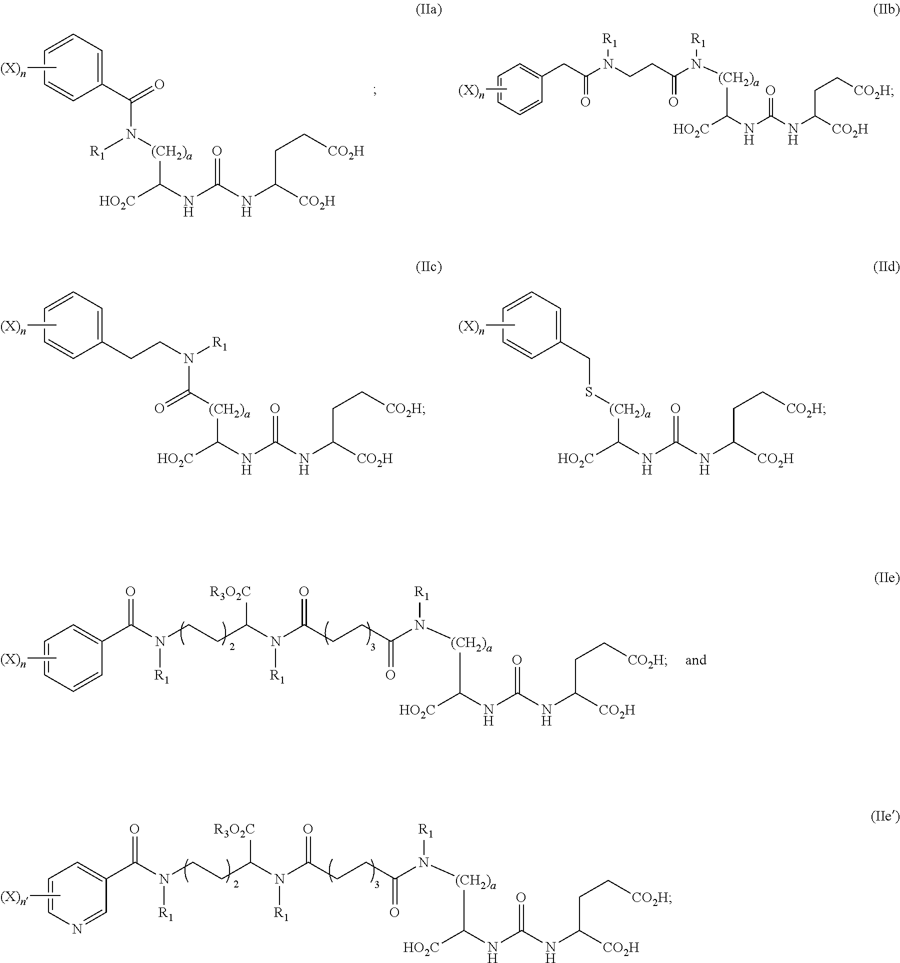
wherein Z, Q, R, R₁, R₃, X, a and n are defined hereinabove;
and stereoisomers and pharmaceutically acceptable salts thereof.

4. The compound of claim 1, wherein X is selected from the group consisting of ¹²⁵I, ¹²³I, ¹³¹I, ²¹¹At ⁷⁷Br, and ^{80m}Br.

5. The compound of claim 1, wherein the compound of formula (I) is selected from the group consisting of:

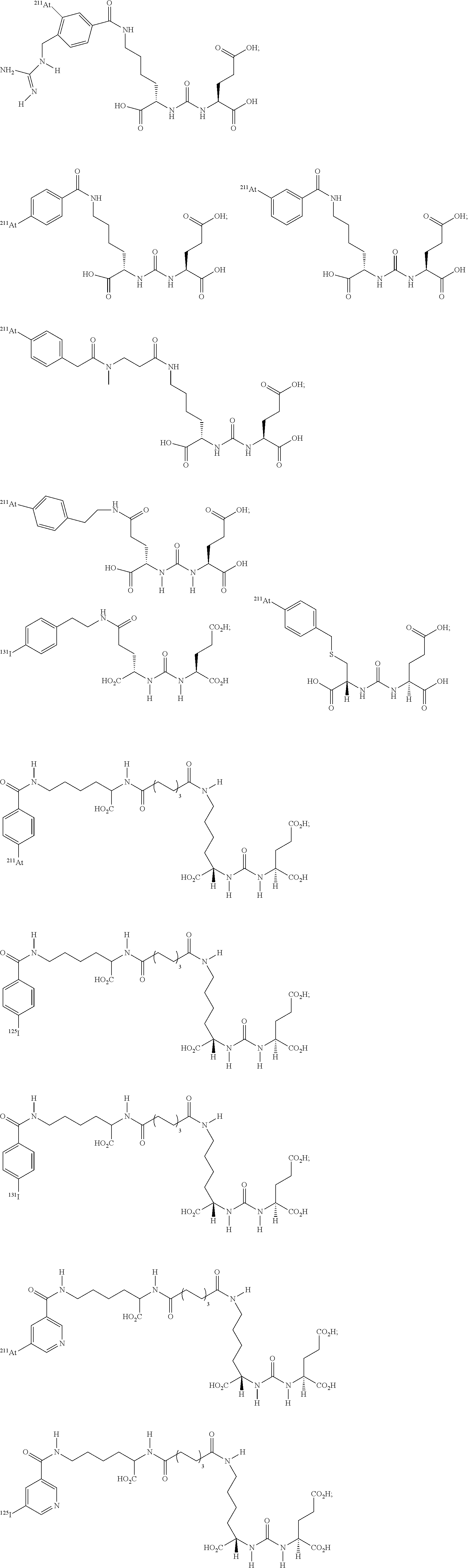

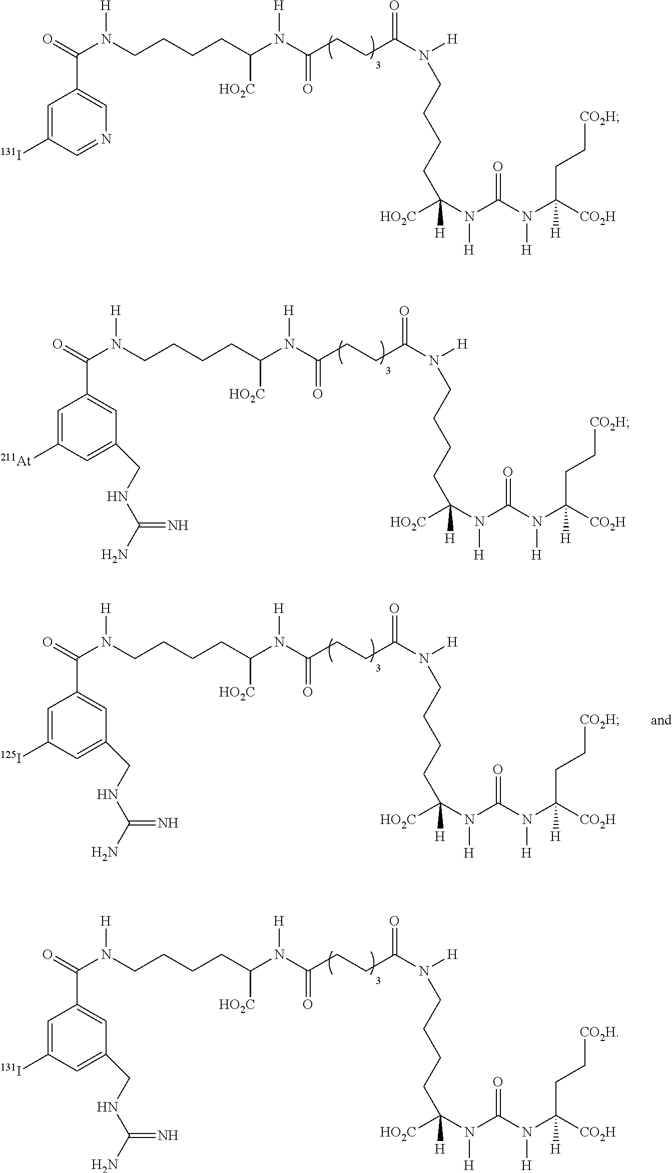

6. The compound of claim 1, wherein the compound of formula (I) is selected from the group consisting of:

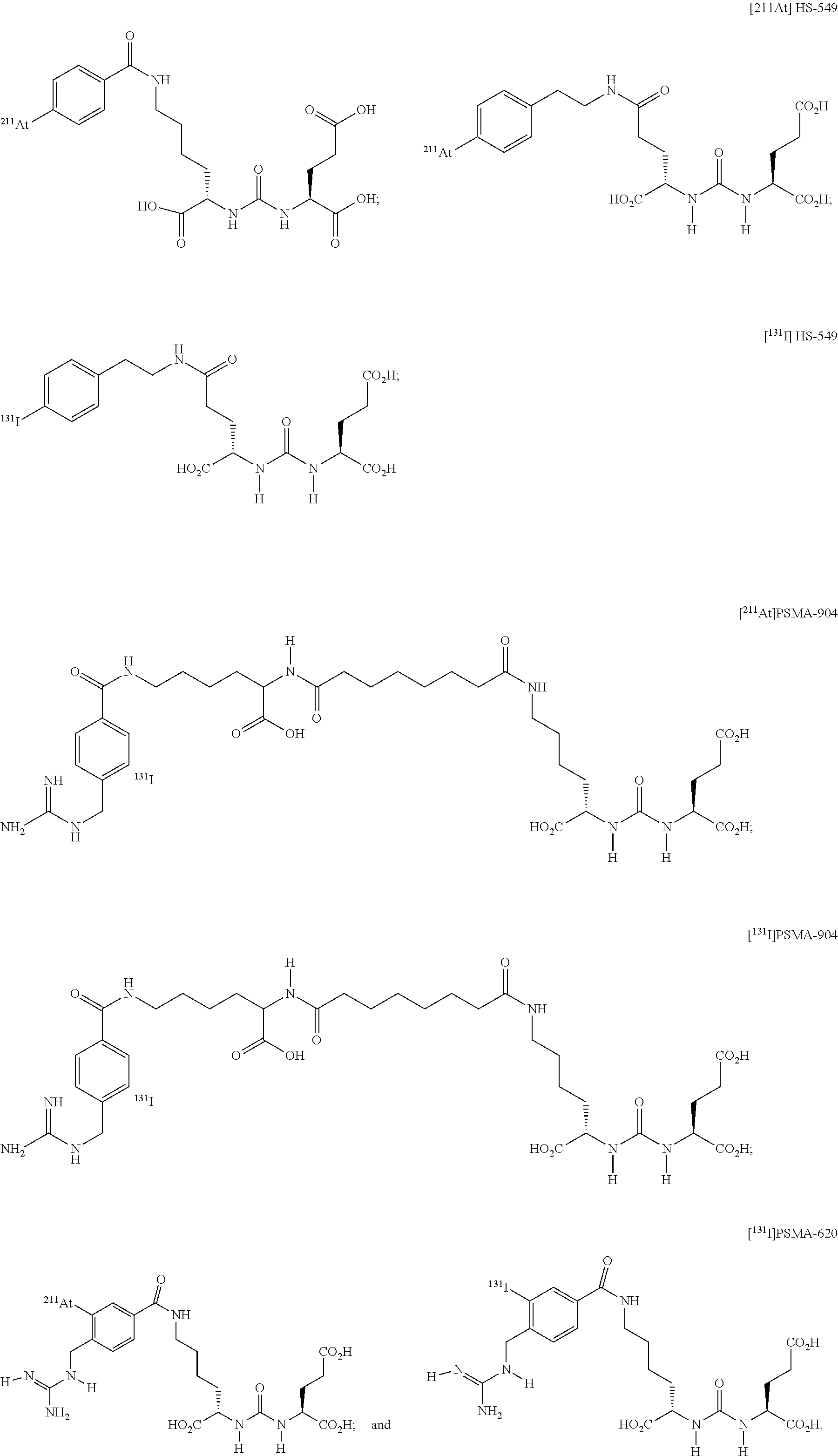

7. A method for treating one or more PSMA expressing tumors or cells, the method comprising contacting the one or more PSMA expressing tumors or cells with an effective amount of a compound of formula (I), the compound of formula (I) comprising: 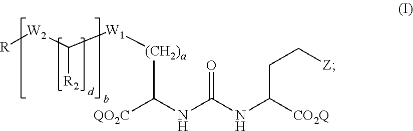
wherein:
Z is tetrazole or CO₂Q;
Q is H or a protecting group;
a is an integer selected from the group consisting of 0, 1, 2, 3, 4, 5, 6, 7, and 8;
W₁ is selected from the group consisting of —C(═O)—NR₁—, —NR₁—C(═O)—, and —S—;
each R₁ is independently H or a C₁-C₆ alkyl;
each R₂ is independently H or —COOR₃;
each R₃ is independently H, C₁-C₆ alkyl, C₆-C₁₂ aryl or C₄-C₁₆ alkylaryl;
b is an integer selected from the group consisting of 0, 1, 2, and 3;
d is an integer selected from the group consisting of 1, 2, 3, 4, 5, 6, 7, and 8;
each W₂ is independently selected from the group consisting of —C(═O)—NR₁— and —NR₁—C(═O)—;
R is selected from the group consisting of: 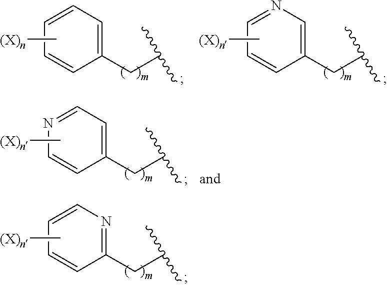
wherein X is Sn(R₄)₃, Si(R₄)₃, Hg(R₄), B(OH)₂, —NHNH₂, —CH₂—NH—C(═NH)—NH₂, a radioisotope of iodine, a radioisotope of astatine, or a radioisotope of bromine;
R₃ is C₁-C₆ alkyl;
m is an integer selected from the group consisting of 0, 1, 2, 3, 4, 5, 6, 7, and 8;
n is an integer selected from the group consisting of 1, 2, 3, 4, and 5;
n′ is an integer selected from the group consisting of 1, 2, 3, and 4;
and stereoisomers and pharmaceutically acceptable salts thereof.

8. The method of claim 7, wherein the compound of Formula (I) is selected from the group consisting of: 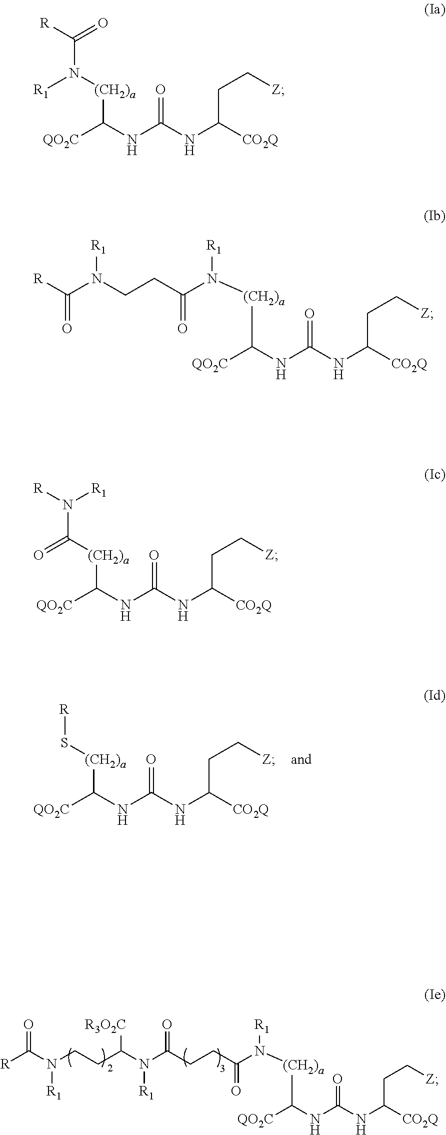
wherein Z, Q, R, R₁, R₃, a are defined as above;
and stereoisomers and pharmaceutically acceptable salts thereof.

9. The method of claim 7, wherein the compound of formula (I) is selected from the group consisting of: 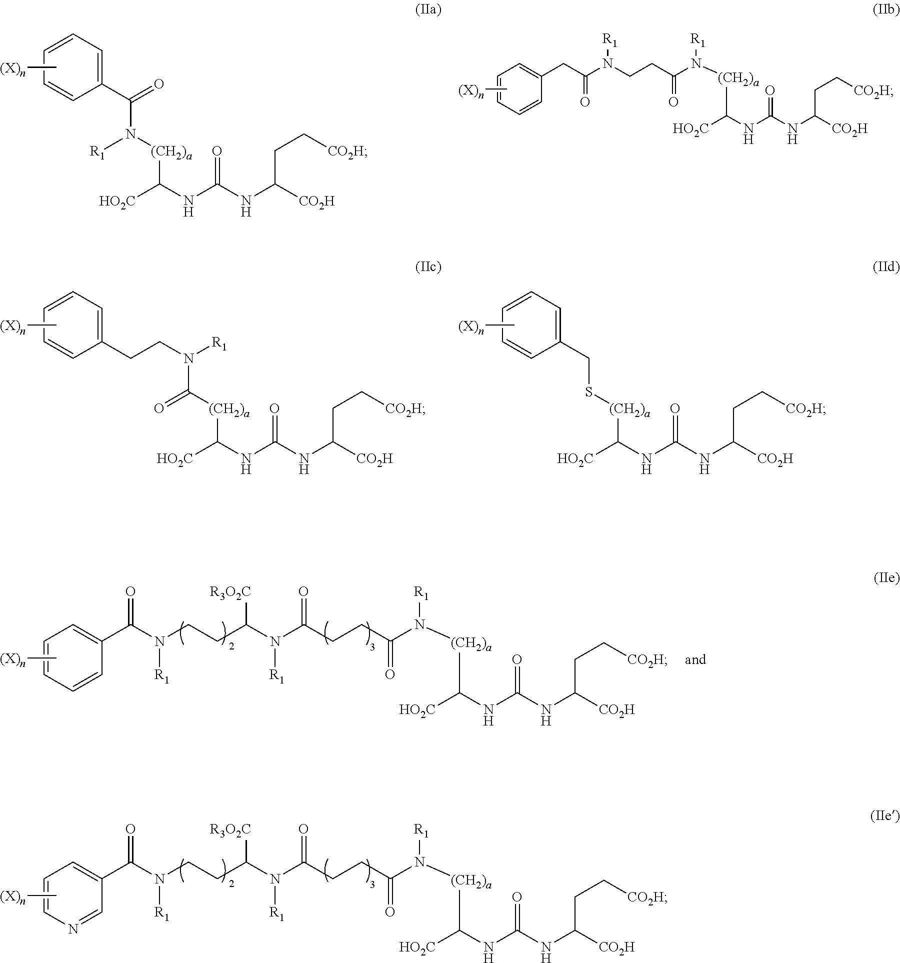
wherein Z, Q, R, R₁, R₃, X, a and n are defined hereinabove;
and stereoisomers and pharmaceutically acceptable salts thereof.

10. The method of claim 7, wherein X is selected from the group consisting of ¹²⁵I, ¹²³I, ¹³¹I, ²¹¹At, ⁷⁷Br, and ^{80m}Br.

11. The method of claim 7, wherein the compound of formula (I) is selected from the group consisting of:

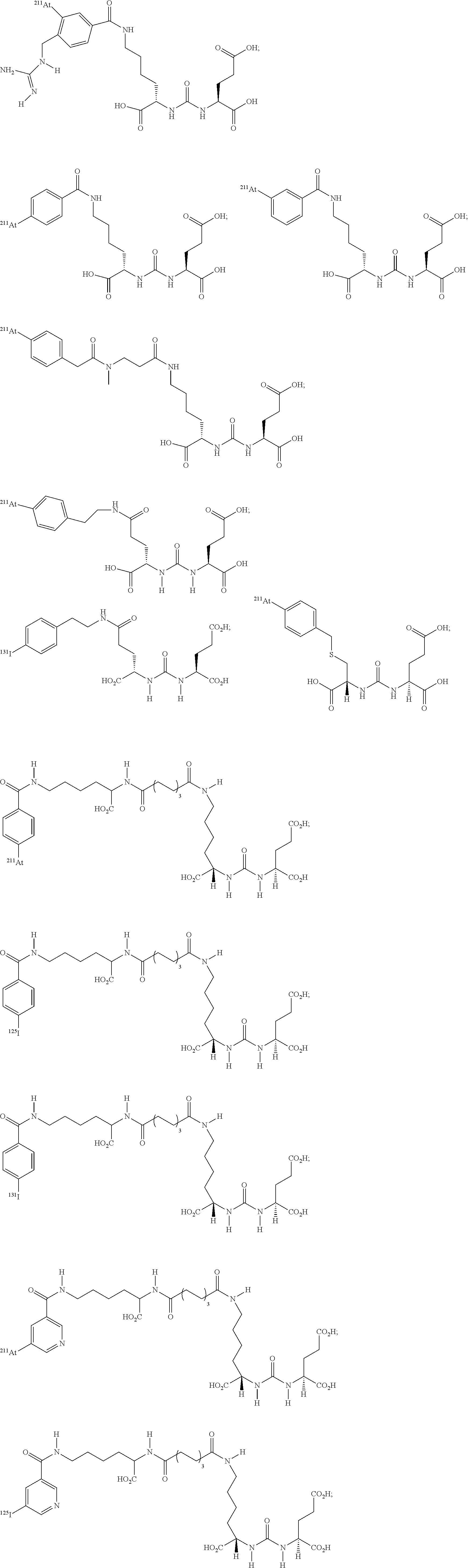

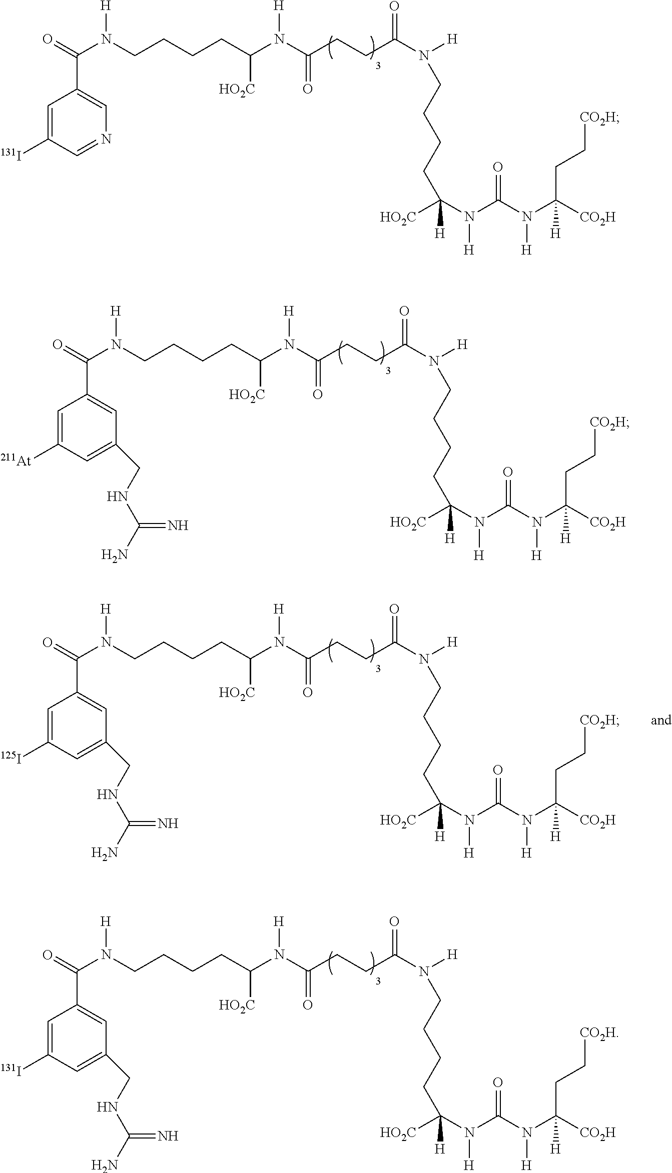

12. The method of claim 7, wherein the compound of formula (I) is selected from the group consisting of:

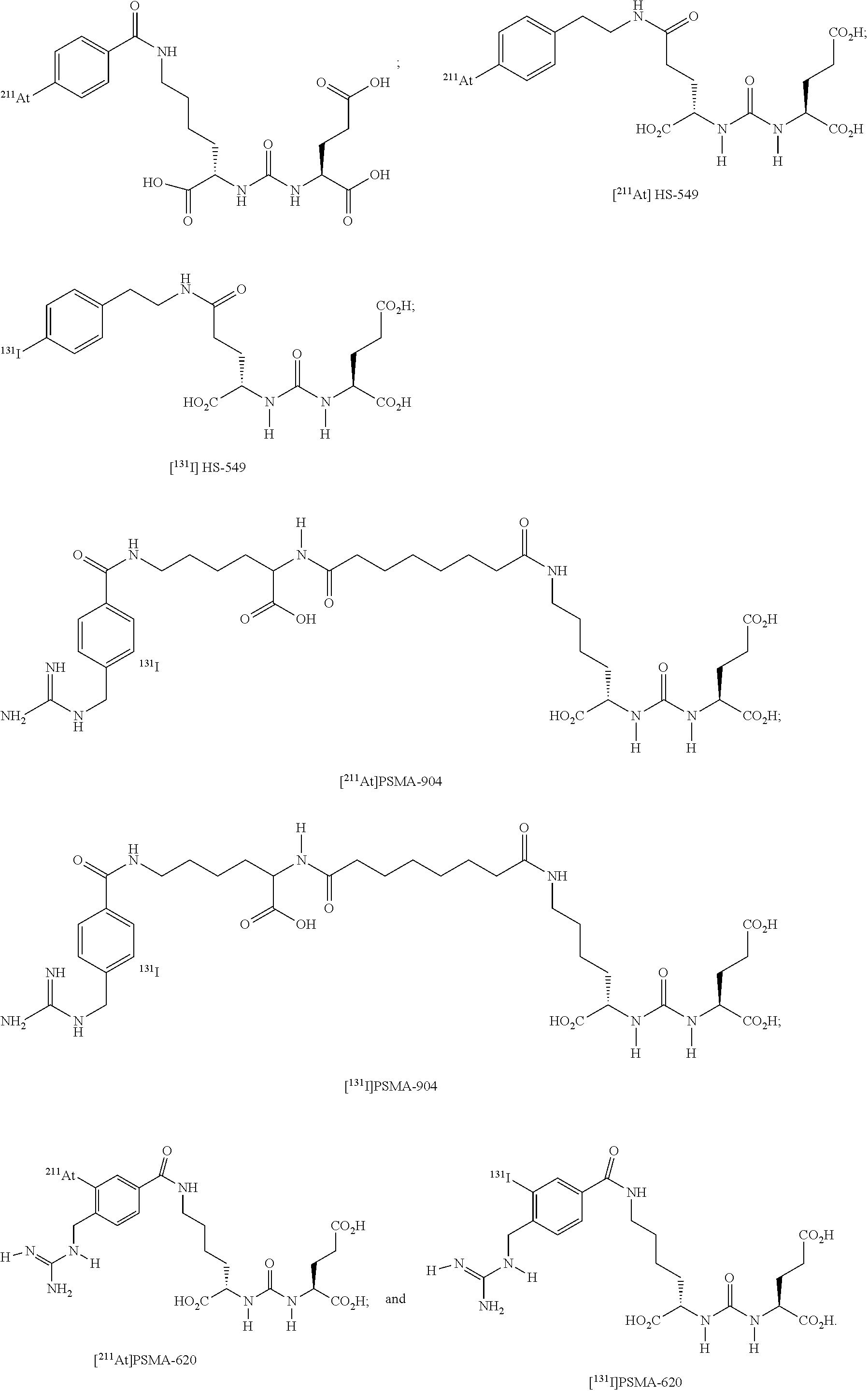

13. The method of claim 7, wherein the one or more PSMA-expressing tumor or cell is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof.

14. The method of claim 7, wherein the one or more PSMA-expressing tumor or cell is a prostate tumor or cell.

15. The method of claim 7, wherein the one or more PSMA-expressing tumors or cells is in vitro, in vivo, or ex vivo.

16. The method of claim 7, wherein the one or more PSMA-expressing tumors or cells is present in a subject.

17. The method of claim 16, wherein the subject is a human.

18. The method of claim 16, wherein the compound of formula (I) is cleared from the subject's kidneys in about 24 hours.

19. The method of claim 7, wherein the method results in inhibition of the tumor growth.

20. The method of claim 7, wherein the compound of formula (I) completely occupies the binding cavity of the PSMA expressing tumors or cells.

21. A pharmaceutical composition comprising a pharmaceutically acceptable carrier and a compound of formula (I), the compound of formula (I) comprising: 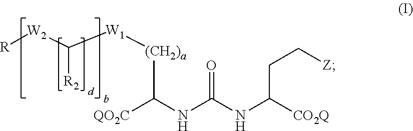
wherein:
Z is tetrazole or CO₂Q;
Q is H or a protecting group;
a is an integer selected from the group consisting of 0, 1, 2, 3, 4, 5, 6, 7, and 8;
W₁ is selected from the group consisting of —C(═O)—NR₁—, —NR₁—C(═O)—, and —S—;
each R₁ is independently H or a C₁-C₄ alkyl;
each R₂ is independently H, —COOH, —COOR₃;
R₃ is independently H, C₁-C₆ alkyl, C₆-C₁₂ aryl or C₄-C₁₆ alkylaryl;
b is an integer selected from the group consisting of 0, 1, 2, and 3;
d is an integer selected from the group consisting of 1, 2, 3, 4, 5, 6, 7, and 8;
each W₂ is independently selected from the group consisting of —C(═O)—NR₁— and —NR₁—C(═O)—;
R is 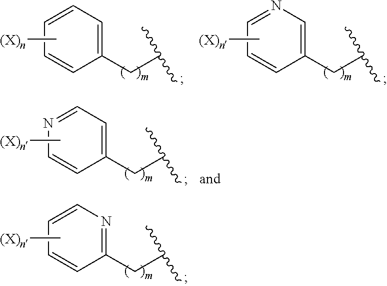
wherein X is Sn(R₄)₃, Si(R₄)₃, Hg(R₄), B(OH)₂, —NHNH₂, —CH₂—NH—C(═NH)—NH₂, a radioisotope of iodine, a radioisotope of astatine, or a radioisotope of bromine;
R₄ is C₁-C₆ alkyl;
m is an integer selected from the group consisting of 0, 1, 2, 3, 4, 5, 6, 7, and 8;
n is an integer selected from the group consisting of 1, 2, 3, 4, and 5;
n′ is an integer selected from the group consisting of 1, 2, 3, and 4;
and acceptable salts thereof.
